(19) United States
(12) Patent Application Publication
(10) Pub. No.: US 2025/0278970 A1
(43) Pub. Date: Sep. 4, 2025

(54) LABORATORY SAFETY ENCLOSURE ASSEMBLY WITH SAFETY FEATURES THEREOF TO CONTROL ACCESS THERETO AND A TRANSPARENT/OPAQUE DISPLAY ON A SASH/WINDOW/DOOR THEREOF

(71) Applicant: H. H. HAWKINS LTD., Surrey (CA)

(72) Inventors: Corey Robert HIEBERT, Surrey (CA); Kevin Philip BOT, Surrey (CA); Adam ZIEGENHAGEL, Surrey (CA)

(73) Assignee: H. H. HAWKINS LTD., Surrey (CA)

(21) Appl. No.: 19/068,973

(22) Filed: Mar. 3, 2025

Related U.S. Application Data
(60) Provisional application No. 63/560,993, filed on Mar. 4, 2024.

(51) Int. Cl. G07C 9/29; B01L 1/00; G06K 7/10

(52) U.S. Cl. CPC G07C 9/29; B01L 1/00; G06K 7/10366; B01L 2300/021; B01L 2300/025

(57) ABSTRACT

There is provided a laboratory safety enclosure assembly including a laboratory safety enclosure with a sash/window/door and a user-identification system to detect a pre-authorized user. The assembly may include a display, and locking mechanism coupled to the sash/window/door and selectively unlockable via the display. A processor restricts access to the interior to users who are pre-authorized, who have viewed and/or completed of a safety training video or course via the display and/or who have completed a safety test via the display with a test score that equals to or exceeds a predetermined threshold. The display may include a digital whiteboard to record/store notes indicia. The display may be selectively 10 transparent during operation thereof and otherwise be opaque to inhibit viewing therethrough. The processor may log and/or time-stamp changing in positioning of the sash/window/door and determine user-specific safety/efficiency information and/or a user-specific safety/efficiency score therefrom.

### BACKGROUND OF THE INVENTION

### Field of the Invention

There is provided a laboratory safety enclosure assembly. In particular, there is provided a laboratory safety enclosure assembly with safety features thereof to control access thereto and a transparent/opaque display on a sash, window and/or door thereof, as well as a method of improving laboratory enclosure safety comprising the same.

Description of the Related Art

U.S. Pat. No. 10,379,607 to Kincaid discloses an environmental chamber having an interior compartment, an augmented display, and a controller is disclosed. The interior compartment is adapted for isolating an experimental setup from an environment external to the interior compartment. The augmented display is positioned to allow a user in the external environment to view the interior compartment and an image generated on the augmented display. The controller generates the image. The image includes information about a component within the interior compartment. The augment display can include a touch-enabled display screen that allows the user to interact with controller.

United States Patent Application Publication No. 2022/0343781 A1 to Kang et al. discloses a method of providing a user-optimized laboratory environment. The method includes receiving user location information and user experiment information from a user terminal. The method includes receiving location information of laboratory equipment and internal environment information of a laboratory from a plurality of sensors. The method includes providing experiment guide information to a user on the basis of the user location information, the user experiment information, the location information of the laboratory equipment, and the internal environment information of the laboratory. The method includes controlling the laboratory equipment on the basis of the user location information, the user experiment information, the location information of the laboratory equipment, and the internal environment information of the laboratory.

United States Patent Application Publication No. 2011/0279265 A1 to Haugen et al. discloses a system for displaying information related to an operational parameter of a biological safety cabinet that may change during ongoing operation thereof. The system comprises a sensor for detecting a prevailing value of the operational parameter at least periodically during operation. The system comprises a display comprising a pictorial graphic having a series of adjacent arcuate segments graphically representing a range of possible values for the operational parameter between a minimum value represented by a first segment, a maximum value represented by a last segment, and incremental intermediate values represented by a plurality of segments intervening between the first and last segments. The system comprises a processor associated with the sensor and with the display for comparing the prevailing value of the operational parameter to the range of possible values and selectively actuate illumination of all, none or another number of the segments to visually signify the prevailing value in relation to the range of values.

### BRIEF SUMMARY OF INVENTION

There is provided, and it is an object to provide, an improved laboratory safety enclosure assembly disclosed herein.

There is provided a laboratory safety enclosure assembly according to one aspect. The assembly includes a laboratory safety enclosure. The assembly includes a user-identification system operatively connected to the laboratory safety enclosure such that operation thereof is a function of the user-identification system detecting a pre-authorized user.

There is also provided a laboratory safety enclosure assembly according to another aspect. The assembly includes a laboratory safety enclosure. The laboratory safety enclosure assembly includes a radio frequency identification (RFID) reader operatively connected to the laboratory safety enclosure such that operation thereof is a function of the RFID reader interacting with one or more RFID tags.

There is additionally provided a laboratory safety enclosure assembly according to a further aspect. The assembly includes a laboratory safety enclosure. The laboratory safety enclosure has an interior and includes a sash, window or door. The laboratory safety enclosure assembly includes a locking mechanism coupled to the sash, window and/or door so as to inhibit access to the interior of the laboratory safety enclosure. The assembly includes a user-identification system operatively connected to the locking mechanism, with unlocking thereof being a function of the user-identification system detecting a pre-authorized user.

There is also provided a laboratory safety enclosure assembly according to yet another aspect. The assembly includes a laboratory safety enclosure. The laboratory safety enclosure has an interior and includes a sash, window and/or door. The assembly includes a locking mechanism coupled to the sash, window and/or door so as to inhibit access to the interior of the laboratory safety enclosure. The assembly includes a radio frequency identification (RFID) reader operatively connected to the locking mechanism, with unlocking thereof being a function of the RFID reader interacting with one or more RFID tags.

There is additionally provided a laboratory safety enclosure assembly according to yet a further aspect. The assembly includes a laboratory safety enclosure with a sash, window and/or door. The assembly includes a display coupled to the sash, window and/or door. The assembly includes a user-identification system operatively connected to the display such that operation thereof is a function of the user-identification system detecting a pre-authorized user.

There is further provided a laboratory safety enclosure assembly according to an additional aspect. The assembly includes a laboratory safety enclosure with a sash, window and/or door. The assembly includes a display coupled to the sash, window and/or door. The assembly includes a radio frequency identification (RFID) reader operatively connected to the display such that operation thereof is a function of the RFID reader interacting with one or more RFID tags.

There is additional provided a laboratory safety enclosure assembly according to yet a further aspect. The assembly includes a laboratory safety enclosure. The laboratory safety enclosure has an interior and includes a sash, window and/or door. The assembly includes a display coupled to the sash, window and/or door. The assembly includes a processor which requires viewing and/or completion of a safety training video or course via the display prior to enabling access to the interior of the laboratory safety enclosure.

There is also provided a laboratory safety enclosure assembly according to another aspect. The assembly includes a laboratory safety enclosure. The laboratory safety enclosure has an interior and includes a sash, window and/or door. The assembly includes a display coupled to the sash, window and/or door. The assembly includes a processor and/or user-identification system to restrict access to the interior of the laboratory safety enclosure to users who are pre-authorized and/or who have viewed and/or completed of a safety training video or course via the display.

There is further provided a laboratory safety enclosure assembly according to an additional aspect. The assembly includes a laboratory safety enclosure with a sash, window and/or door. The assembly includes a display coupled to the sash, window and/or door. The assembly includes a processor and/or user-identification system to require satisfactory completion of a safety test via the display prior to enabling access to the interior of the laboratory safety enclosure.

There is yet also provided a laboratory safety enclosure assembly according to another aspect. The assembly includes a laboratory safety enclosure. The laboratory safety enclosure has an interior and includes a sash, window and/or door. The assembly includes a display coupled to the sash, window and/or door. The assembly includes a processor and/or user-identification system to restrict access to the interior of the laboratory safety enclosure to users who are pre-authorized, who have viewed and/or completed of a safety training video or course via the display and/or who have completed a safety test via the display with a test score that equals to or exceeds a predetermined threshold.

There is additionally provided a laboratory safety enclosure assembly according to another aspect. The assembly includes a laboratory safety enclosure. The laboratory safety enclosure has an interior and includes a sash, window and/or door. The assembly includes a locking mechanism coupled to the sash, window and/or door so as to inhibit access to the interior of the laboratory safety enclosure. The assembly includes a display operatively connected to the locking mechanism, with the locking mechanism being selectively unlockable via the display.

There is also provided a laboratory safety enclosure assembly according to a further aspect. The assembly includes a laboratory safety enclosure with a sash, window and/or door. The assembly includes a display coupled to the sash, window and/or door. The display is selectively or at least partially transparent. The display includes a graphical user interface with a medium via which notes indicia may be created.

There is further provided a laboratory safety enclosure assembly according to yet another aspect. The assembly includes a laboratory safety enclosure with a sash, window and/or door. The assembly includes a display coupled to the sash, window and/or door. The display is selectively at least partially transparent and comprises a digital whiteboard.

There is yet also provided a laboratory safety enclosure assembly according to a further aspect. The assembly includes a laboratory safety enclosure. The laboratory safety enclosure has an interior and includes a sash, window and/or door. The assembly includes a display coupled to the sash, window and/or door. The display is selectively at least partially transparent during operation thereof. The display hides or conceals at least in part the interior of the laboratory safety enclosure when in a privacy mode thereof.

There is further provided a laboratory safety enclosure assembly according to an additional aspect. The assembly includes a laboratory safety enclosure. The laboratory safety enclosure has an interior and includes a sash, window and/or door. The assembly includes a display coupled to the sash, window and/or door. The display selectively enters an operational mode in which the interior of the laboratory safety enclosure is visible therethrough. The display selectively enters a privacy mode in which the display inhibits viewing of the interior of the laboratory safety enclosure therethrough.

There is further provided a laboratory safety enclosure assembly according to another aspect. The assembly includes a laboratory safety enclosure. The laboratory safety enclosure has an interior and includes a sash, window and/or door. The assembly includes a position-sensing device that measures positioning of the sash, window and/or door and emits one or more signals based thereon. The assembly includes a display via which access to the interior of the laboratory safety enclosure is enabled. The assembly includes a processor operatively connected to the position-sensing device and the display. The processor logs each change in positioning of the sash, window and/or door. The processor logs time-in and time-out information and/or the time at which a logging-in event occurs and the time at which a logging-off event occurs.

There is also provided a laboratory safety enclosure assembly according to an additional aspect. The assembly includes a laboratory safety enclosure with a sash, window and/or door. The assembly includes a position-sensing device that measures positioning of the sash, window and/or door and emits one or more signals based thereon. The assembly includes a display via which features thereof are accessible upon logging-in with user information. The assembly includes a processor operatively connected to the position-sensing device and the display. The processor logs and/or obtains data relating to each change in positioning of the sash, window and/or door and correlates the data so logged with the user information so as to determine user-specific efficiency information, user-specific safety information, a user-specific safety score, and/or a user-specific efficiency score.

There is further provided a laboratory safety enclosure assembly according to another aspect. The assembly includes a laboratory safety enclosure with a sash, window and/or door. The assembly includes a user-identification system to detect a pre-authorized user. The assembly includes a display coupled to the sash, window and/or door. The display includes a digital whiteboard to record/store notes indicia thereon. The display is selectively transparent during operation thereof. The display is selectively opaque to inhibit viewing therethrough in a privacy mode thereof. The assembly includes a locking mechanism coupled to the sash, window and/or door. The locking mechanism is selectively unlockable via the display. The assembly includes a processor operatively connected to the user-identification system, the display and the locking mechanism. The processor restricts access to the interior of the laboratory safety enclosure to users who are pre-authorized, who have viewed and/or completed of a safety training video or course via the display and/or who have completed a safety test via the display with a test score that equals to or exceeds a predetermined threshold. The processor logs and/or time-stamps changes in positioning of the sash, window and/or door and determines a user-specific efficiency score therefrom.

There is additionally provided a laboratory safety enclosure assembly according to yet another aspect. The assembly includes a laboratory safety enclosure. The laboratory safety enclosure has an interior and includes a sash, window and/or door. The assembly includes a radio frequency identification (RFID) reader that interacts with one or more RFID tags to enable access to the interior of the laboratory safety enclosure. The assembly includes a display coupled to the sash, window and/or door. The display includes a digital whiteboard to record/store notes indicia thereon. The display is selectively transparent during operation thereof. The display is selectively opaque to inhibit viewing therethrough in a privacy mode thereof. The assembly includes a locking mechanism coupled to the sash, window and/or door. The locking mechanism is selectively unlockable via the display. The assembly includes a processor to restrict access to the interior of the laboratory safety enclosure to users who are pre-authorized, who have viewed and/or completed of a safety training video or course via the display and/or who have completed a safety test via the display with a test score that equals to or exceeds a predetermined threshold. The processor logs and/or time-stamps changes in positioning of the sash, window and/or door and determines a user-specific efficiency score therefrom.

There is further provided a laboratory safety enclosure assembly according to another aspect. The assembly includes a laboratory safety enclosure. The assembly includes a person-detecting device coupled to the laboratory safety enclosure. The assembly includes a memory. The assembly includes a processor operatively connected to the person-detecting device and memory. The processor restricts access to the assembly unless i) a person is detected via the person-detecting device; ii) the person is authorized and/or within a database in said memory; and iii) the person is certified to use the assembly.

There is also provided a laboratory safety enclosure assembly according to a further aspect. The assembly includes a display. The assembly includes a memory. The assembly includes a processor operatively connected to the display and the memory. The processor restricts access thereto unless a person is authorized and certified to use the assembly as indicated within a database in said memory. If the person as determined by the processor is not authorized and/or certified, the processor requires that that the person both i) view training materials via the display as part of a certification process and ii) satisfactorily pass a test or quiz via the display as part of the certification process, to obtain access to the assembly.

There is also provided a method of improving laboratory enclosure safety according to one aspect. The method includes coupling a user-identification system to a laboratory safety enclosure such that operation thereof is a function of the user-identification system detecting a pre-authorized user.

There is further provided a method of improving laboratory enclosure safety according to another aspect. The method includes coupling a locking mechanism to a sash, window and/or door of a laboratory safety enclosure so as to inhibit access to an interior of the laboratory safety enclosure. The method includes operatively connecting a user-identification system to the locking mechanism, with unlocking thereof being a function of the user-identification system detecting a pre-authorized user.

There is further provided a method of improving laboratory enclosure safety according to an additional aspect. The method includes coupling a display to a sash, window and/or door of a laboratory safety enclosure. The method includes operatively connecting a user-identification system to the display such that operation thereof is a function of the user-identification system detecting a pre-authorized user.

There is further provided a method of improving safety of a laboratory safety enclosure assembly according to one aspect. The method includes requiring that a person be detected by the assembly via a person-detecting device as a first condition precedent and/or first safety layer/barrier for obtaining access to the assembly. The method includes requiring that the person be authorized and/or within a database in a memory of the assembly as a second condition precedent and/or second safety layer/barrier for obtaining access to the assembly. The method includes requiring that the person be certified as a third condition precedent and/or third safety layer/barrier for obtaining access to the assembly.

There is additionally provided a method of improving safety of a laboratory safety enclosure assembly according to another aspect. The method includes requiring that a person be authorized and/or within a database in a memory of the assembly as a first condition precedent for obtaining access to the assembly. The method includes requiring that that the person be certified as a second condition precedent for obtaining access to the assembly. If the person is not certified, the method includes requiring that that the person both i) view training materials via a display of the assembly as part of a certification process and ii) satisfactorily pass a test or quiz via said display as part of the certification process, to obtain access to the assembly.

There is also provided a method of determining a rental rate for a laboratory safety enclosure assembly according to one aspect. The method includes measuring via a position sensing device positioning of a sash, window and/or door of a laboratory safety enclosure of the assembly in real-time and emitting one or more signals based thereon. The method includes logging via the position-sensing device each change in positioning of the sash, window and/or door and/or obtaining data via the position sensing device related thereto. The method includes correlating via a processor the data so logged with user information. The method includes determining the rental rate of the assembly as a function of and/or proportionate to said data so correlated.

There is also provided a method of determining a rental rate for a laboratory safety enclosure assembly according to one aspect. The method includes measuring via a position-sensing device positioning of a sash, window and/or door of a laboratory safety enclosure of the assembly in real-time and emitting one or more signals based thereon. The method includes logging via the position-sensing device each change in positioning of the sash, window and/or door and/or obtaining data via the position sensing device related thereto. The method includes correlating via a processor the data so logged with user information so as to determine user-specific efficiency information, user-specific safety information, a user-specific safety score, and/or a user-specific efficiency score. The method includes determining the rental rate of the assembly as a function of and/or proportionate to said user-specific efficiency information, user-specific safety information, user-specific safety score and/or user-specific efficiency score.

It is emphasized that the invention relates to all combinations of the above features, even if these are recited in different claims.

Further aspects and example embodiments are illustrated in the accompanying drawings and/or described in the following description.

### BRIEF DESCRIPTION OF DRAWINGS

The accompanying drawings illustrate non-limiting example embodiments of the invention: FIG. 1 is a front, top, right side perspective view of a laboratory safety enclosure assembly according to one aspect, the assembly including an HVAC system, the assembly including a laboratory safety enclosure operatively connected to the HVAC system, with the laboratory safety enclosure including a sash shown in a partially open position and including a display coupled to the sash thereof, with the display being at least partially opaque so as to inhibit viewing therethrough;

FIG. 2 is a front, top, right side perspective view thereof with the HVAC system thereof not being shown, with the sash being in a closed and locked position, and with a safety training video being shown via the display;

FIG. 3 is a front, top, right side perspective view thereof, with the display being at least partially transparent so as to facilitate viewing therethrough to the interior of the laboratory safety enclosure, and with the display showing interactive experiment information or indicia thereon;

FIG. 4 is a front, top, right side perspective view of the laboratory safety enclosure assembly thereof on a laboratory bench, with a person shown adjacent thereto, with the display of the assembly being opaque, and with the assembly including one or more visual indicators shown via the display to warn the person of an alarm relating to a safety incident/event/risk;

FIG. 5 is a schematic block diagram of the laboratory safety enclosure assembly of FIG. 1, including various features thereof;

FIG. 6 is a flow chart for the laboratory safety enclosure assembly of FIG. 1, illustrating an algorithm for enabling access to the laboratory safety enclosure (or interior or display thereof) according to one aspect;

FIG. 7 is a flow chart for the laboratory safety enclosure assembly of FIG. 1, illustrating an algorithm for enabling access to the laboratory safety enclosure (or interior or display thereof) according to another or complementary aspect;

FIG. 8 is a flow chart for the laboratory safety enclosure assembly of FIG. 1, illustrating an algorithm for enabling access to the laboratory safety enclosure (or interior or display thereof) according to yet another or complementary aspect;

FIG. 9 is a schematic block diagram of a graphical user interface of the laboratory safety enclosure assembly of FIG. 1 according to one non-limiting aspect;

FIG. 10A is a schematic block diagram of a plurality of software applications and widgets for the laboratory safety enclosure assembly of FIG. 1; and

FIG. 10B is a schematic block diagram of an additional plurality of software applications for the laboratory safety enclosure assembly of FIG. 1.

### DESCRIPTION OF THE PREFERRED EMBODIMENTS

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Referring to the drawings and first to FIG. 1, there is shown a laboratory safety enclosure assembly, in this non-limiting embodiment a fume hood assembly 40. However, this is not strictly required and the laboratory safety enclosure assembly may comprise other types of enclosure assemblies in other embodiments, such as a biosafety cabinet assembly, for example.

Fume hood assembly 40 includes a laboratory safety enclosure, in this non-limiting example a fume hood 42. However, here too this is not strictly required and other types of laboratory safety enclosures may be provided in other embodiments, such as a biosafety enclosure or cabinet of a biosafety cabinet assembly. Fume hood 42 is a rectangular prism in outer shape in this non-limiting embodiment; however this is not strictly required. The fume hood has a top 44, a bottom 46 spaced-apart from the top thereof, a rear 48, a front 50 spaced-apart from the rear thereof, a first or left side 52 and a second or right side 54 spaced-apart from the left side thereof. The top, bottom, front and rear of fume hood 42 extend between the left and right sides of the fume hood. Top 44, bottom 46, rear 48, front 50 and sides 52 and 54 of the fume hood are rectangular in this non-limiting example; however here too this is not strictly required. Fume hood 42 includes a base 55 adjacent bottom 46. The fume hood has and/or encloses an interior 56 within which one or more experiments 57 may be conducted, such as on or adjacent the base thereof.

Still referring to FIG. 1, fume hood assembly 40 includes in this non-limiting embodiment a ventilation system, in this example a heating, ventilation and air conditioning (HVAC) system 58. However, this is not strictly required and in other embodiments the fume hood assembly is connectable to the HVAC system. The HVAC system couples to fume hood 42, in this non-limiting example via top 44 of the fume hood. HVAC system 58 is in fluid communication with interior 56 of the fume hood. The HVAC system selectively provides an airflow through and/or draws air/gas from the interior of the fume hood away therefrom as shown by arrows 60 and 62. HVAC system 58 includes an air-displacement device 64 (e.g. a fan) via which air/gas is directed through ducting 66 and away from fume hood 42 so as to remove toxic and/or noxious fumes and the like therefrom. The HVAC system may provide a variable flow rate of gas through the fume hood. HVAC systems, including their various parts and functionings, are known per se and HVAC system 58 will accordingly not be described in further detail.

Fume hood assembly 40 includes a processor 68. The processor may be locally situated or remotely situated. Processor 68 in this non-limiting embodiment couples to fume hood 42 adjacent front 50 and side 54 thereof.

Fume hood assembly 40 in this non-limiting example includes an alarm system 69. The alarm system includes in this embodiment a speaker 71. Processor 68 is operatively connected to alarm system 69 (and/or speaker thereof), with the processor selectively causing notifications or alarms to be sounded via the alarm system upon determining that a safety event/incident/risk has occurred or is occurring.

Fume hood assembly 40 in this non-limiting embodiment includes one or more and in this example a plurality of sensors in communication with processor 68 to this end, with the sensors being arranged to promote safety and/or facilitate performing one or more experiments 57 therewithin. Non-limiting examples of sensors may include a temperature sensor 70, a humidity sensor 72, and an airflow sensor 74 of an airflow monitoring system 75. The sensors are in fluid communication with interior 56 of fume hood 42. Sensors 70, 72 and 74 measure temperature, humidity and airflow data of fume hood assembly 40 and/or of fluid or gas located within the interior of the fume hood thereof, and output signals 70A, 72A and 74A in response thereto. Each signal as herein described for fume hood assembly 40 may be transmitted wirelessly or via a wired connection/conduit. Processor 68 operatively connects to the sensors and receives signals 70A, 72A and 74A and determine temperature, humidity and airflow flow rates based thereon. Sensors 70, 72 and 74 couple to fume hood 42 and are positioned adjacent front 50 and side 52 of the fume hood in this non-limiting embodiment; however, this is not strictly required.

Still referring to FIG. 1, fume hood assembly 40 in this non-limiting embodiment includes an image sensor, in this example a camera 76. The camera is positioned to view interior 56 of fume hood 42. Camera 76 is positioned within the interior of the fume hood in this non-limiting embodiment and may thus be referred to as an internal camera; however, this is not strictly required. The camera captures video and/or images of the interior of fume hood, such as the status or progress of one or more experiments 57 and outputs said video/images to a remote or local processor, in this example processor 68 operatively connected thereto. The camera may be referred to as an additional sensor of fume hood assembly 40. Camera 76 couples to fume hood 42 adjacent top 44 of the fume hood in this non-limiting embodiment; however, this is not strictly required. The camera is positioned to capture images/video of experiments 57 and/or of a region adjacent or near base 55 of fume hood 42 in this example.

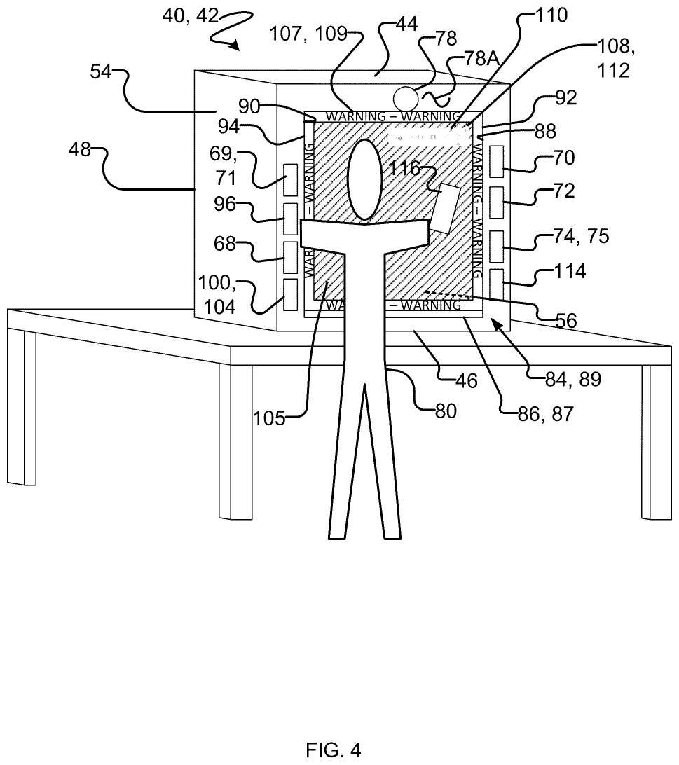

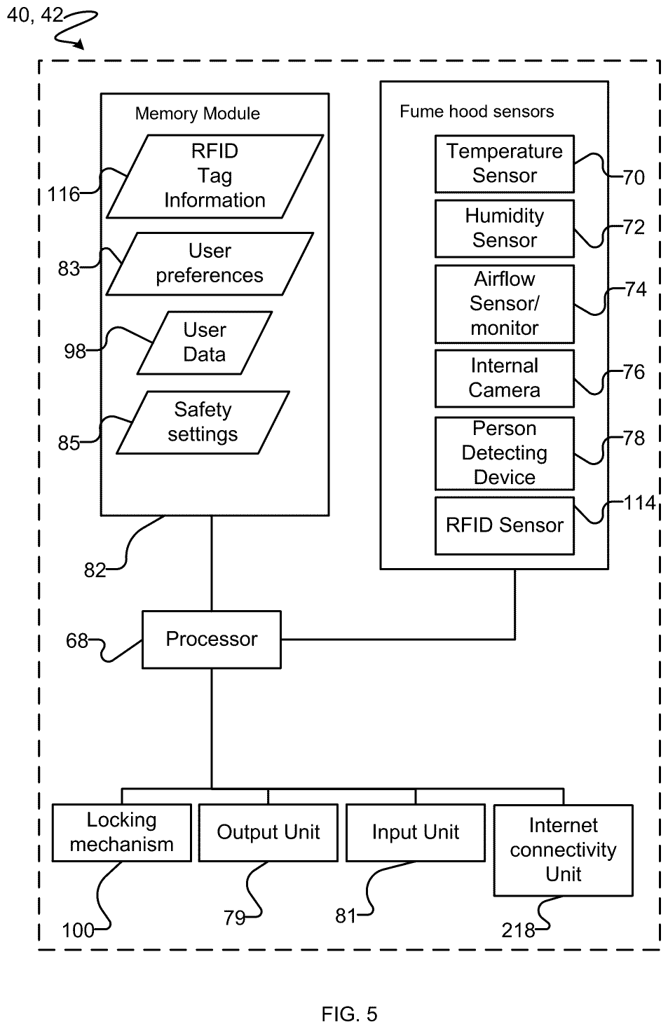

Fume hood assembly 40 includes in this non-limiting embodiment a person-detecting device 78. The person-detecting device operatively connects to fume hood 42, in this non-limiting example being positioned adjacent top 44 and front 50 of the fume hood and between sides 52 and 54 of the fume hood. Referring to FIG. 4, person-detecting device 78 detects the presence of a user or person 80 near or adjacent fume hood 42 and emit a signal 78A in response thereto. Processor 68 is operatively connected to the person-detecting device so as to receive the signal and determine in response thereto whether the person is sufficiently near or adjacent to fume hood 42. The processor determines that the person is proximate to the fume hood when signal 78A is equal to or exceeds a predetermined threshold. Person-detecting device 78 may comprise in one non-limiting embodiment a sensor, such as an occupancy sensor, a passive infrared sensor, a millimeter wave (mmWave) sensor, a camera sensor and/or combination thereof. However, this is not strictly required and the person-detecting device may comprise a camera in other non-limiting embodiments with facial-recognition software and/or technology to predict/determine the identity of person 80 and determine whether they are stored in a memory module 82 (or database or memory thereof) of fume hood assembly 40 seen in FIG. 5. Processor 68 operatively connects to the memory module.

Still referring to FIG. 5, memory module 82 may comprise local or remote data storage. The memory module operatively connects to processor 68 and stores information thereon. This information in this non-limiting example includes a database of information or user information/data 98 comprising a list of persons who are authorized/registered to use/operate fume hood 42 and/or a list of users who are certified to use/operate the fume hood. The latter may comprise users who have successfully completed a certification process as discussed further below. Memory module 82 may additionally include stored thereon/therewithin user preferences 83 specific to a given user, as well as safety settings 85, for example.

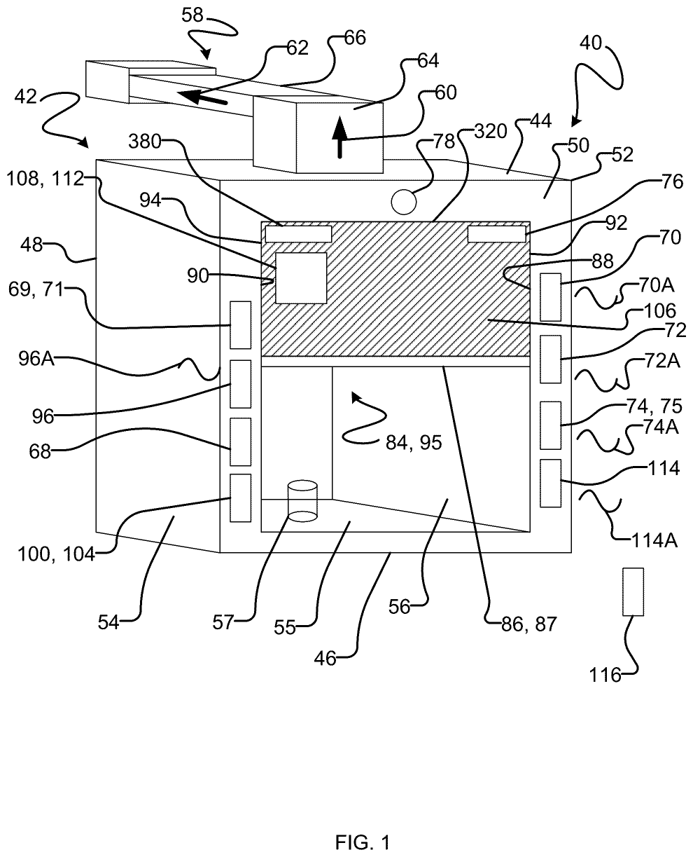

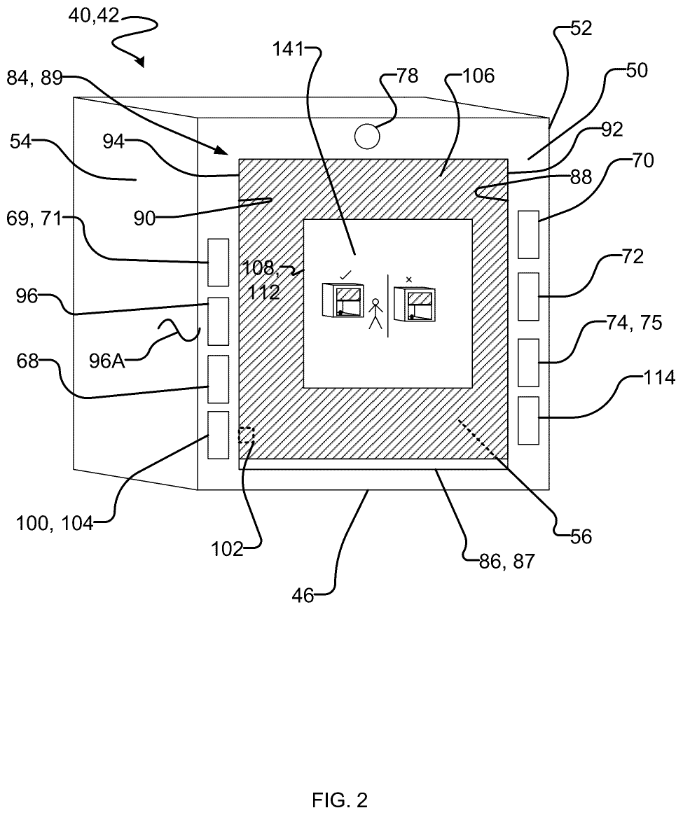

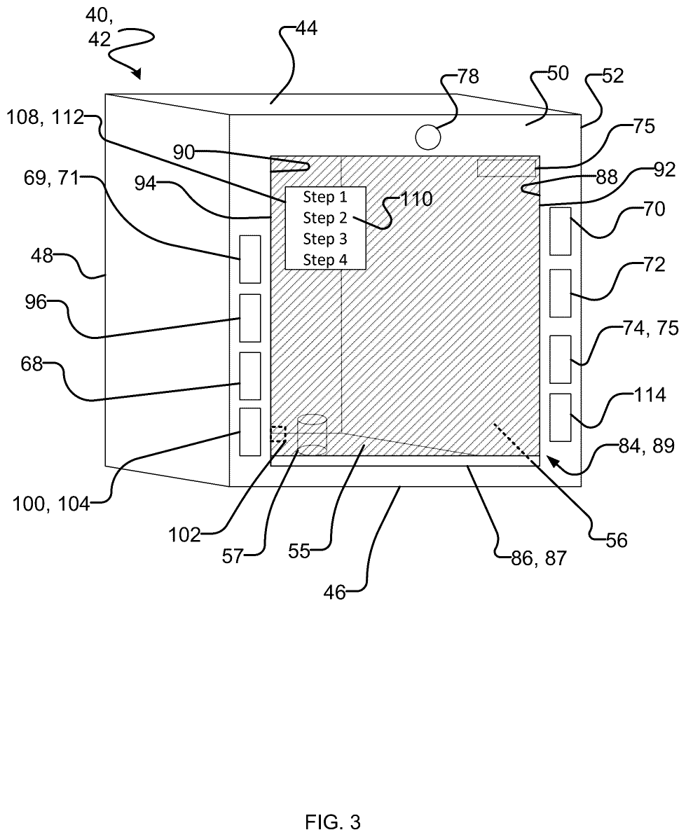

Referring now to FIG. 1, fume hood assembly 40 includes a sash 84, which may be said to be part of fume hood 42. The sash may be referred to as a window and/or door, or in the alternative, instead of a sash, the fume hood assembly may comprise a window and/or door. Sash 84 is transparent at least in part and is made of glass in this non-limiting example; however, this is not strictly required and the sash may be made of other transparent materials in other examples such as clear plastic or the like. The sash has a first or open position seen in FIG. 1, in which access to interior 56 of fume hood 42 is enabled/promoted/facilitated. FIG. 1 may be said to show a partially open position of sash 84, with the interior of the fume hood being fully exposed/accessible via front 50 of fume hood 42 is a fully open position (not shown) of the sash. The partially or fully open positioned of the sash may facilitate setting up, adjusting and/or directly/tangibly interacting with one or more experiments 57, for example. Sash 84 is moveable from the open (or partially open) position to a second or closed position seen in FIGS. 2 and 3, in which access to interior 56 of fume hood 42 is inhibited. The following is a non-limiting embodiment which achieves this functionality.

Sash 84 in this non-limiting example includes a handle 86 coupled to and extending outwards/forwards from adjacent bottom 87 thereof. Fume hood 42 includes one or more elongate members, in this example one or more spaced-apart frame members, in this case a pair of frame members 88 and 90 via which sides 92 and 94 of the sash are linearly displaceable and/or moveably engageable. In this non-limiting example the frame members may comprise a pair of rails and/or have elongate grooves within which the sides of sash 84 slidably engage. The sash in this non-limiting embodiment slidably couples to the rails and is moveable from open and closed positions thereby. Sash 84 may comprise a slidable window which is moveable from the closed position thereof seen in FIGS. 2 and 3, in which access to interior 56 of fume hood 42 is inhibited, to its open position seen in FIG. 1, in which access to the interior of the fume hood is promoted and/or facilitated. The sash has a front 95 extending upwards from bottom 87 thereof and extending between sides 92 and 94 thereof. The front of sash 84 is rectangular in this non-limiting embodiment.

As seen in FIG. 2, fume hood assembly 40 includes a position-sensing device 96. The position-sensing device may be comprise a sensor, such as a position sensor; however, this is not strictly required and the device may be a camera in other embodiments, for example. Position-sensing device 96 measures positioning of sash 84 and emits one or more signals 96A based thereon. Processor 68 is operatively connected to the position-sensing device.

Positioning sash 84 in its at least partially open position as seen in FIG. 1 for an extended period of time, may lower or reduce the energy efficiency of fume hood assembly 40 and/or increase the risk of safety incidents or accidents such as harmful fumes being inhaled by person 80 seen in FIG. 4. Positioning-sensing device 96 operates in real-time in this non-limiting example and outputs data indicative of the positioning of the sash.

Processor 68 in this non-limiting example logs and time stamps in real-time each change in positioning of sash 84 and correlates the data so logged/received with user information or data 98 stored in memory module 82 seen in FIG. 5 so as to determine a user-specific efficiency information, user-specific safety information, a user-specific safety score (e.g. a sash that is closed a greater percentage of the time, such as above a predetermined threshold of time, may result in safer laboratory practice) and/or a user-specific efficiency score. This information and/or score may provide the user and/or a supervising authority (such as an instructor/teacher/professor or laboratory supervisor/manager/director) with feedback on how to better operate fume hood assembly 40 going forward.

The data to be logged when a user is logged in may include: user name data, time logged in and out data, sash position data with a timestamp associated with each change thereof, and data from the alarm monitor of the HVAC system via a building automation and control network (BACnet) protocol. Fume hood assembly 40 is arranged to promote collection of relevant data to enable third parties, such as institutions, to better determine and/or quantify which users/user-groups are being more or less energy efficient and/or to provide a means for billing for fume hood use.

Regarding the latter, there may thus be provided a method of determining a rental or charge rate for fume hood assembly 40. The method may include measuring via position-sensing device 96 seen in FIG. 1, positioning of sash 84 of the fume hood assembly in real-time and emit one or more signals 96A based thereon. The method may include logging via the position-sensing device each change in positioning of the sash and/or obtaining data via the position sensing device related thereto. The method may include correlating via processor 68 the data so logged with user information or data 98 seen in FIG. 5. The method may include determining the rental rate of fume hood assembly 40 as a function of and/or proportionate to said data so correlated.

For example, a user who positions sash 84 seen in FIG. 1 in a fully or at least partially open position for a prolonged/extended period of time which exceeds a predetermined amount of time or median amount of time for a group or class, may be operating fume hood assembly 40 in a less efficient and/or less safe manner and be charge at a higher rental or charge rate. For example, positioning of the sash, window and/or door for a period of time which exceeds a predetermined time threshold may result in an increased said rental rate and positioning of the sash, window and/or door for a period of time which is less than said predetermined time threshold may result in a discounted said rental rate.

Fume hood assembly 40 as herein described may thus function to incentivize more energy-efficient and safer laboratory practices. The fume hood assembly as herein described may thus result in fewer safety incidents and/or accidents, thereby promoting laboratory safety, as well as promoting energy efficiency.

Conversely, fume hood assembly 40 may reward and/or offer a discounted rental or charge rate to users who promote positioning of sash 84 in its closed position seen in FIG. 3 relative to a predetermined threshold or medium amount of time for the group or class. The method may include tracking said user information by requiring a user to login to display 106 in order to have access to fume hood assembly 40. The method may include logging alarm actuation/triggering information and correlating the same with user information or data 98 seen in FIG. 5 to determine the user-specific efficiency information, user-specific safety information, user-specific safety score and/or user-specific efficiency score. The method may include determining the rental or charge rate of the fume hood assembly as a function of and/or proportionate to user-specific efficiency information and/or user-specific efficiency score.

In addition or alternatively, the method may include monitoring the extent to which one or more alarms are triggered while the user is logged in and/or operating fume hood assembly 40 and increasing or discounting the rental rate based on thereon. The method may include adjusting the rental rate of the fume hood assembly so determined based on the extent to which one or more alarms are actuated while the user is logged in and/or operating the fume hood assembly. Thus or in the alternative, the method may include adjusting the rental rate of the fume hood assembly so determined based on user-specific safety information and/or a user-specific safety score.

As a further addition or alternative, there may also thus be provided a method of improving fume hood assembly safety comprising monitoring positioning of sash 84 of fume hood 42 via position-sensing device 96 and emitting one or more signals 96A seen in FIG. 1 based thereon. The method may include receiving via processor 68 to receive the one or more signals and logging and time stamping via the processor each change in positioning of the sash base on thereon. The method may include correlating via the processor the data so logged with user information (e.g. obtained via login/logout information) so as to determine the user-specific efficiency information, user-specific safety information, user-specific safety score and/or user-specific efficiency score.

Referring to FIG. 1, processor 68 selectively enables or inhibits access to fume hood 42. The following is one non-limiting embodiment which achieves this functionality.

Fume hood assembly 40 includes a locking mechanism 100. The locking mechanism couples to fume hood 42. Locking mechanism 100 in this non-limiting example is near or adjacent bottom 46, side 54 and front 50 of the fume hood; however, this is not strictly required and may be positioned elsewhere in other embodiments. The locking mechanism is selectively operatively connectable to sash 84. Locking mechanism 100 in this non-limiting embodiment couples to the sash so as to inhibit access to interior 56 of fume hood 42. The locking mechanism has a first, open or unlocked position seen in FIG. 1 in which sash 84 is selectively moveable upwards or downwards relative to frame members 88 and 90 so as to enable access to interior 56 of fume hood 42.

Locking mechanism 100 has a second or locked position, seen in FIGS. 2 and 3, in which movement of the sash relative to the frame members is inhibited. The locking mechanism may comprise any type of locking member(s) which selectively operatively engage(s) with sash 84 so as to inhibit linear displacement thereof when the locking mechanism is actuated to its locked position thereof. As seen in FIG. 2, locking mechanism 100 in this non-limiting example includes a catch 102 that selectively operatively engages with the sash in the locked position. The locking mechanism selectively engages with sash 84 in its closed position in this non-limiting embodiment; however, this limitation is not strictly required.

Processor 68 operatively couples to locking mechanism 100. The processor inhibits access to interior 56 of fume hood 42 via selective actuation of the locking mechanism. Locking mechanism 100 in this non-limiting embodiment includes and/or is selectively actuated via an actuator, in this non-limiting example a servomotor 104. The servomotor is in communication with processor 68, via which sash 84 may be selectively unlocked. The processor enables access to the fume hood by actuating locking mechanism 100 to the unlocked position.

Processor 68 may cause automatic locking of sash 84 if person 80 seen in FIG. 4 is not proximate to fume hood 42 as determined by person-detecting device 78. The processor may inhibit unlocking of the sash if the person is not proximate the fume hood as determined by the person-detecting device.

As seen in FIG. 1, fume hood assembly 40 includes a display 106. The display comprises an electronic device for the visual presentation of data and/or information. Display 106 is coupled to sash 84. Alternatively, the sash may be said to comprise the display or vice versa. The display in this example couples to front 89 of sash 84 and extends between sides 92 and 94 of the sash in this non-limiting embodiment. Display 106 in this non-limiting example is rectangular and co-extensive with the sash; however, in either case this is not strictly required.

As seen in FIG. 3, the display is (or is capable of being) selectively transparent at least in part so as to facilitate or enable viewing therethrough into interior 56 of fume hood 42. Display 106 comprises a graphical user interface (GUI) 108. The display is touch sensitive. GUI 108 conveys experiment data thereon, including one or more information, status and/or indicia related thereto, as generally shown by numeral 110. The GUI includes a medium, in this non-limiting example a touchscreen 112. Display 106, including transparent display, graphical user interfaces, and touchscreens, including their various parts and functionings, are known per se and the general parts and functionings of the display, GUI 108 and touchscreen 112 will accordingly not be described in further detail.

Processor 68 operatively couples to the display. Display 106 enables one or more persons 80 seen in FIG. 4 to login with user information or data 98 seen in FIG. 5. The processor in this non-limiting embodiment logs time-in and time-out information for a specific user, and/or the time at which a logging-in event occurs and the time at which a logging-off event occurs. Display 106 seen in FIG. 3 provides customized or customizable settings based on a given said user, as shown by user preferences 83 in FIG. 5. This may also be a part of the user information.

Referring back to FIG. 2, access to interior 56 of fume hood 42 is enabled via display 106 in one non-limiting embodiment. Processor 68 enables access to the fume hood by enabling an expanded set of features of the display to be accessible.

As seen in FIG. 1, fume hood assembly 40 includes a user-identification system, in this non-limiting example an identification sensor and one or more objects to which the sensor is interactable: in this non-limiting embodiment the sensor comprises a radio frequency identification (RFID) reader 114 and the one or more objects comprise one or more RFID cards or tags as shown by RFID tag 116. However, this is not strictly required and other types of user-identification systems or sensors (or systems/sensors with an ID factor or other type of sensing device with the ability to identify a specific person/user) may be used in other embodiments such as: a fingerprint, iris and/or facial user-identification system, scanner and/or detection device (e.g. camera with artificial intelligence (AI) and/or facial recognition technology/software). RFID reader 114 in this non-limiting example is operatively connected to fume hood 42, in this example coupling to front 50 of the fume hood. The RFID reader is positioned to facilitate person 80 seen in FIG. 4 interacting therewith. RFID reader 114 in this non-limiting example is near or adjacent bottom 46 and side 52 of fume hood 42 as seen in FIG. 1; however, this is not strictly required. RFID tags 116 may comprise pre-authorized RFID tags in one non-limiting embodiment. RFID reader 114 operatively connects to fume hood 42 such that operation thereof is a function of the RFID reader interacting with the one or more RFID tags. The following is a non-limiting embodiment that achieves this functionality.

RFID reader 114 outputs a signal 114A upon interacting with one or more RFID tags 116. The RFID reader is operatively connected to processor 68, with the processor receiving said signal. The processor determines whether to enable access to fume hood 42 based on said information. Processor 68 enables operation of and/or actuates the fume hood and/or components thereof or expanded features of fume hood assembly 40, upon receiving signal 114A according to one embodiment. Locking mechanism 100 operatively connects to RFID reader 114 and processor 68 selectively actuates the locking mechanism to its unlocked position seen in FIGS. 1 and 4 thereafter according to another embodiment. The unlocking of the locking mechanism is a function of the RFID reader interacting with one or more RFID tags 116 in this non-limiting example.

In addition or alternatively, processor 68 after receiving signal 114A, determines whether the one or more RFID tags is authorized. The processor enables operation of and/or actuates fume hood 42 and/or components thereof or expanded features of fume hood assembly 40, upon received determining that the one or more RFID tags is authorized according to another non-limiting embodiment. In this embodiment processor 68 selectively actuates locking mechanism 100 to its unlocked position seen in FIGS. 1 and 4 upon determining that said one or more RFID tags is authorized. Within this embodiment, display 106 may not be strictly required and enabling operation of fume hood assembly 40 may comprise RFID reader 114 interacting with RFID tag 116 and sending signal 114A to processor 68, with the processor enabling operation of locking mechanism 100 in response thereto so to enable movement of sash 84 to its open position seen in FIG. 1.

According to one non-limiting embodiment, RFID reader 114 operatively connects to display 106 such that operation thereof is a function of the RFID reader interacting with one or more RFID tags 116. Processor 68 enables operation of an expanded set of features of the display upon receiving signal 114A and/or determining that the one or more RFID tags is authorized. Within this embodiment, locking mechanism 100 may not be strictly required and enabling operation of fume hood assembly 40 may comprise RFID reader 114 interacting with RFID tag 116, sending signal 114A to processor 68, with the processor enabling operation of display 106 in response thereto.

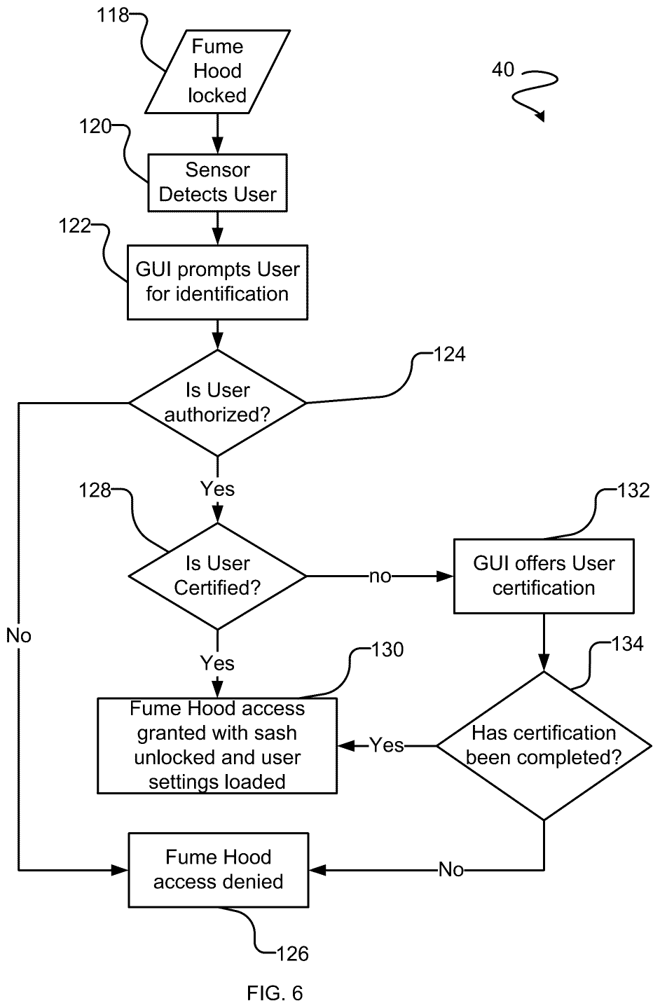

FIG. 6 shows an algorithm for enabling access to the fume hood (or interior or display thereof) according to one non-limiting embodiment of fume hood assembly 40. In operation, the fume hood is locked by default as shown by box 118. Upon the person-detecting device or sensor detecting a user as shown by box 120, the display or GUI thereof prompts the user for identification as shown by box 122. This may be in the form of the user presenting RFID card or tag 116 seen in FIG. 1 near or adjacent RFID reader 114. Referring back to FIG. 6, the method includes next determining whether the user is authorized as shown by box 124. This may involve processor 68 receiving signal 114A from RFID reader 114 seen in FIG. 1 and processing the signal to determine whether the user associated with the tag is within a user database or user data 98 of memory module 82 seen in FIG. 5.

If the processor determines that no authorized user is associated with the RFID card or tag based on the information stored within the memory module, then the processor inhibits and/or denies access to the fume hood as shown by box 126. This denial of access may comprise maintaining sash 84 in its closed and locked position via locking mechanism 100 as seen in FIG. 2. In addition or alternatively, this denial of access may comprise inhibiting access to controls and/or features of fume hood assembly 40 otherwise accessible via display 106. For example, this may comprise keeping the user logged-out/locked-out of the software applications and control systems accessible/adjustable via the display. The term “software application” and “application software” as herein described may be used interchangeably.

Referring back to FIG. 6, if the processor determines that the user associated with the RFID tag or card is within the memory module and/or authorized, the method next includes determining whether the user is certified as shown by box 128. This may involve processor 68 processing signal 114A seen in FIG. 1 to determine whether the user associated with the RFID card or tag has a completed safety/training/certification program/orientation or has otherwise completed comparable steps, as indicated by information within the user database or user data 98 of memory module 82 seen in FIG. 5.

If the processor determines that the user associated with the RFID card or tag has completed a certification program or is authorized indicated to be certified based on the user data in the memory module, then the processor enables or facilitates access to the fume hood assembly as shown by box 130 in FIG. 6. This may include actuating servomotor 104 of locking mechanism 100 to unlock sash 84 seen in FIG. 1 so as to enable access to interior 56 of fume hood 42. In addition or alternatively, this may include enabling access via display 106 to controls and/or features of fume hood assembly 40. For example, this may comprise enabling the user to login and have access to software applications and control systems accessible/adjustable via the display. Processor 68 may load user settings 85 corresponding the RFID card or tag of the user.

As seen in FIG. 6, if the processor determines the user associated with the RFID card or tag is not certified as indicated by the memory module, then the display or graphical user interface (GUI) prompts the user to complete a user safety/training/certification program/orientation via the display as shown box 132. Fume hood assembly 40 next determines via the processor thereof whether the user has successfully completed the safety/training/certification program/orientation as shown by box 134, with outputs from the display to this end being received by the processor to be processed so as to determine the same. If the processor determines that the user has successfully completed safety/training/certification program/orientation via the display, then the processor enables or facilitates access to the fume hood assembly as shown by box 130 and discussed further above. If the processor determines that the user so authorized is not certified and/or has not successfully completed safety/training/certification program/orientation via the display, then the processor inhibits access or denies to the fume hood as shown by box 126.

There is also provided a method of improving fume hood safety. The method may include coupling RFID reader 114 seen in FIG. 1 to fume hood 42 such that operation thereof is a function of the RFID reader interacting with one or more RFID cards or tags 116. The method may include coupling locking mechanism 100 to sash 84 of the fume hood so as to inhibit access to interior 56 of the fume hood. The method may include operatively connecting RFID reader 114 to the locking mechanism, with unlocking thereof being a function of the RFID reader interacting with one or more RFID cards or tags 116. In addition or alternatively, the method may include coupling display 106 to sash 84 of fume hood 42. The method may include operatively connecting RFID reader 114 to the display such that operation thereof is a function of the RFID reader interacting with one or more RFID cards or tags 116. The method may thus include operatively connecting display 106 to locking mechanism 100, with the locking mechanism being selectively unlockable via the display.

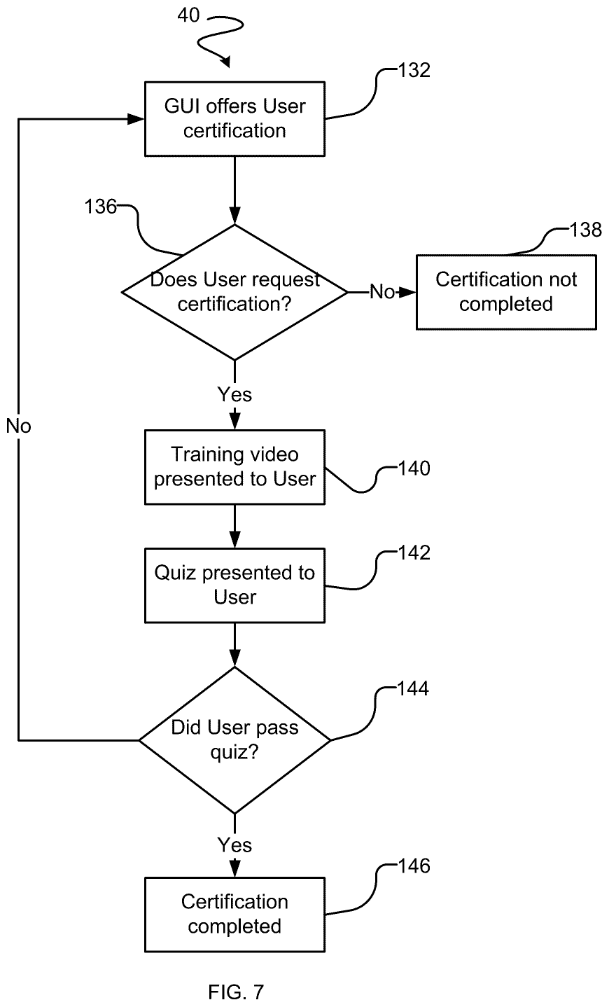

FIG. 7 shows an algorithm for enabling access to the fume hood (or interior or display thereof) according to another or complementary aspect, in this case a non-limiting example for determining whether the safety/training/certification program/orientation has been successfully completed so as to result in a certified user. The method includes the display or GUI offering the user (who may or may not be an authorized user according to different embodiments) the ability to obtain user certification as shown by box 132. Fume hood assembly 40 may include next determining via the processor thereof whether the user requests certification, as shown by box 136. This may comprise offering/suggesting-to the user via a touchscreen prompt to initiate the certification process. If the user rejects or fails to accept the invitation to complete the certification process, the processor determines that certification for that specific user is not completed as shown by box 138. Fume hood access may thus be denied as shown by box 126 in FIG. 6.

Referring back to FIG. 7, if the processor determines that the user has accepted the invitation to complete the certification process, the method in this non-limiting embodiment includes next causing training materials, in this non-limiting example a training video, to be presented to the user via the display as shown by box 140. The training video 141 is shown schematically in FIG. 2.

Upon completing the training materials and referring back to FIG. 7, the method may in one non-limiting embodiment include presenting a test or quiz to the user via the display as shown by box 142. The processor tabulates and/or analyzes the user-provided answers for the quiz and determines whether the user has satisfactorily passed the quiz as shown by box 144. If no, the algorithm/method may be offer the user the opportunity to repeat the certification process once more as shown by box 132.

If the processor determines that the user has satisfactorily passed the quiz, the processor determines that certification is complete for the user associated with the RFID card or tag as shown by box 146. The processor may also update user data 98 in memory module 82 so as to indicate/record that this user is now a certified user.

As a further addition or alternative, the method described above may thus include configuring display 106 seen in FIG. 2 to require viewing thereon and/or completion of safety training video 141 or course prior to enabling access to interior 56 of fume hood 42. The method may include restricting access to the interior of the fume hood to users who are pre-authorized and/or who have viewed and/or completed of the safety training video or course via the display. The restricting step may include coupling locking mechanism 100 to sash 84 which promotes positioning of the sash in its closed position seen in FIG. 2. The restricting step may include selectively unlocking the locking mechanism via processor 68 to enable or facilitate access to interior 56 of fume hood 42.

The method may include configuring display 106 to require satisfactory completion of a safety test via the display prior to enabling access to the interior of the fume hood. The method may include restricting access to interior 56 of fume hood 42 to users who are pre-authorized, who have viewed and/or completed of the safety training video or course via the display and who have completed the safety test via the display with a test score that equals to or exceeds a predetermined threshold.

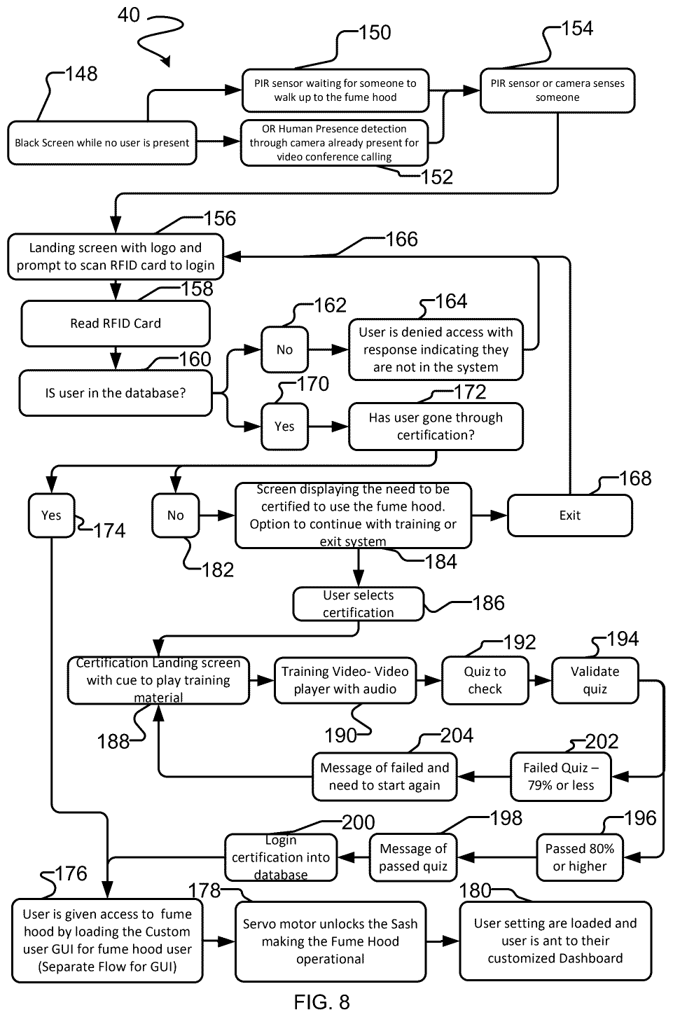

FIG. 8 shows an algorithm for enabling access to the fume hood (or interior or display thereof) according to a further non-limiting embodiment of fume hood assembly 40. The algorithm, fume hood assembly and/or method may comprise five layers of safety, stages, requirements and/or conditions precedent in order to obtain access to the fume hood assembly (and/or the fume hood or expanded features of the display thereof). In operation and according to this non-limiting embodiment, the display is opaque, in this example black, while no user is determined to be present as shown by box 148 in FIG. 8. Fume hood assembly 40 remains on stand-by, with a person-detecting device in the form of i) a passive infrared (PIR) sensor waiting for someone to walk up to the fume hood as shown box 150 and/or ii) a camera (e.g. already presented for video conferencing calling) to detect/identify a human presence as shown by box 152.

The method includes next determining via the sensor or camera, that someone is present or adjacent/near the fume hood as shown by box 154. The method may include identifying a specific user using the camera and facial-recognition software and/or technology.

In addition or alternatively, when fume hood assembly 40 so determines via the processor thereof that the person is present, the display conveys a user-prompt to scan an RFID card or tag to login to the system as shown by box 156. Processor 68 seen in FIG. 4 thus receives signal 78A from person-detecting device 78 and in response thereto operatively prompts person 80 to scan RFID card or tag 116 via RFID reader 114. The requirement that a user first be detected by fume hood assembly 40 may be said to comprise a first layer of safety, criteria and/or condition precedent to obtain access to the fume hood assembly. This may be in the form of a landing screen in which a proprietary/company logo is also displayed; however, this is not strictly required.

Referring back to FIG. 8, the method includes reading information contained within the RFID card or tag via the RFID reader as shown by box 158. This information is next communicated by RFID reader 114 via signal 114A seen in FIG. 1 to processor 68. In this non-limiting embodiment, the RFID reader reads user information contained within the RFID card or tag and communicates the user information to the processor. The processor determines whether to enable access to the fume hood based on said information. As seen in FIG. 8, this may include in this non-limiting embodiment the processor determining whether said user information is in the database stored in memory or a database thereof, as shown by box 160. There may thus be provided a method of improving fume hood assembly safety that includes identifying a specific user by scanning an RFID tag, which may be associated with said specific user.

If the processor determines that the user associated with the RFID card or tag is not of record or within the database of authorized users of fume hood assembly 40 as shown by box 162, then the processor takes no action and/or inhibits access to fume hood and the user is denied with a response conveyed via the display indicating that they are not in the system as shown by box 164. The user is thus prompted to scan an RFID card anew once more 166 and/or the display may exit as shown by box 168 and/or be rendered opaque or black once more. The requirement that the user be authorized and/or within the database in the memory of fume hood assembly 40 may be said to comprise a second layer of safety, criteria and/or condition precedent to obtain access to the fume hood assembly.

If the processor determines that the user associated with the RFID card or tag is of record or in the database stored in memory as shown by box 170, then the processor next determines whether the user information comprises a user who has successfully completed a safety training and/or certification process as shown by box 172.

If yes as shown by box 174, then the processor causes access to the fume hood to be enabled. In this case, if the processor both determines that the user information is in the database and determines that a user associated with said user information has successfully completed a safety training and/or certification process, then the processor enables access to the fume hood as shown by box 176. The requirement that the user be certified may be said to comprise a third layer of safety, criteria and/or condition precedent to obtain access to the fume hood assembly. The processor provides access to the fume hood by loading, in this non-limiting example, a custom GUI on the display for fume hood use according to this non-limiting embodiment. In addition or alternatively, the processor enables access to the fume hood by enabling the user to successfully login into fume hood assembly 40 via the display and/or enable an enlarged access to programs and features of fume hood assembly and/or the display upon determining that the above criteria have been met.

The algorithm includes unlocking the sash to make the fume hood operational as shown box 178, in this example by the processor actuating the servomotor to move the locking mechanism to its unlocked position. The process may next include user settings being loaded and the user being sent to their customized dashboard, as shown by box 180.

If the processor determines that the user information is in the database but that the corresponding user has not successfully completed the safety training and/or certification process as shown by box 182, then fume hood assembly 40 conveys a screen on the display indicating the need to be certified to use the fume hood as shown by box 184. This may also include an option to continue with the training or exit the system. The processor prompts the user to continue with training via the display. If the user selects the exit option such as via a touchscreen interaction with the display and as shown by box 168, then the user may be returned to the landing screen with a prompt to scan an RFID card to login anew as shown by box 156.

If the user selects the continue with training option such as via a touchscreen interaction with the display and as shown by box 186, then fume hood assembly 40 provides training materials, in this non-limiting example via a certification landing screen with a cue or prompt to play the training materials as shown by box 188. If the user presses the play option, fume hood assembly 40 initiates training which may include training materials and/or a safety training video shown via the display as shown by box 190, which may include a video player with audio. The processor in this non-limiting example requires viewing and/or completion of the safety training video or course via the display prior to enabling access to the interior of the fume hood. The processor in this non-limiting embodiment restricts access to the interior of the fume hood to users who are pre-authorized and/or who have viewed and/or completed of a safety training video or course via the display. The requirement that the user view training materials via the display as part of the certification process may be said to comprise a fourth layer of safety, criteria and/or condition precedent to obtain access to the fume hood assembly.

In addition or alternatively, fume hood assembly 40 may next and as part of the training process, require that the user complete a quiz as shown by box 192. The method may next include validating the quiz as shown by box 194 including tabulating via the processor a score thereof. The method may include requiring that the user achieve a test or quiz score that equals to or exceeds a minimum test score threshold as shown by 196, in this non-limiting example a result of 80% or higher. Fume hood assembly 40 via the processor thereof prompts uncertified users to take a quiz, with satisfactory completion thereof requiring the user to meet or exceed a predetermined test score and/or threshold.

If such a test score or result is determined to have been achieved, then the display may in this non-limiting embodiment convey a message via the display that the quiz has been passed as shown by box 198. Fume hood assembly 40 next logins certification into the database as shown by box 200, in this case updating its memory to associate the RFID card or tag of the user with the status of being certified. The user is then given access to the fume hood as shown by boxes 176, 178 and 180 and discussed above. If the processor both determines that said user information is in the database and determines that a user associated with said user information has successfully completed a safety training and/or certification process, then the processor thus enables access to the fume hood.

If a test result is achieved which is less than the minimum test score threshold, in this non-limiting example comprising 79% or less as shown by box 202, then fume hood assembly 40 in this non-limiting embodiment conveys a message via the display that the user failed to meet the minimum test score threshold as shown by box 204 and that the quiz must be performed anew for example. Thus, if the processor determines that the user information is in the database but corresponds to a user who has not as yet successfully completed a safety training and/or certification process, then the processor prompts the user to continue with training via the display.

Processor 68 seen in FIG. 2 thus in this non-limiting embodiment enables access to fume hood 42 to the user upon determining that the training has been completed and/or the user has completed the quiz with a test score that meets or exceeds the predetermined test score and/or threshold. The processor thus requires satisfactory completion of a safety test via display 106 prior to enabling access to the interior of the fume hood. Processor 68 thus in this non-limiting example restricts access to interior 56 of fume hood 42 to users who are pre-authorized, who have viewed and/or completed of a safety training video or course via the display and who have completed a safety test via the display with a test score that equals to or exceeds a predetermined threshold.

The requirement that the user view satisfactorily pass a test or quiz via display 106 seen in FIG. 2 as part of the certification process may be said to comprise a fifth layer of safety, criteria and/or condition precedent to obtain access to fume hood assembly 40. The user information or data 98 seen in FIG. 5 may thus include one or more safety criteria associated with the user, such as whether the login user is in the database or of record, whether the training has been completed and/or the quiz satisfactory completed. In one non-limiting embodiment to this end, processor 68 causes locking mechanism 100 seen in FIG. 2 to unlock in this non-limiting embodiment upon determining that the above safety criteria have been met.

Referring to FIG. 3, there may be provided a method of improving safety of fume hood assembly 40 comprising rendering fume hood 42 transparent at least in part when the safety criteria is satisfied. The method may include providing a safety criteria comprising answering a safety quiz displayed on sash 84 or display 106 coupled to front 95 of the sash. The method may include providing a safety criteria comprising displaying a video to the user. The method may include displaying the video on the sash or on display coupled to the front thereof. The method may include as part of the safety criteria, determining via processor 68 whether a specific user is within a database of authorized said users.

In addition or alternatively, a method is provided comprising detecting whether person 80 is proximate to fume hood 42 as seen in FIG. 2, automatically locking sash 84 of the fume hood if the user is not proximate to the fume hood and unlocking the sash when one or more of the above safety criteria is satisfied. The method may include within the detecting step, coupling person-detecting device 78 near or adjacent fume hood 42. The method may include within the detecting step, determining via processor 68 that person 80 is proximate to the fume hood when signal 78A of person-detecting device 78 is equal to or exceeds a predetermined threshold. The method may include inhibiting viewing through sash 84 when the user is not proximate to fume hood 42: this may include by rendering display 106 opaque.

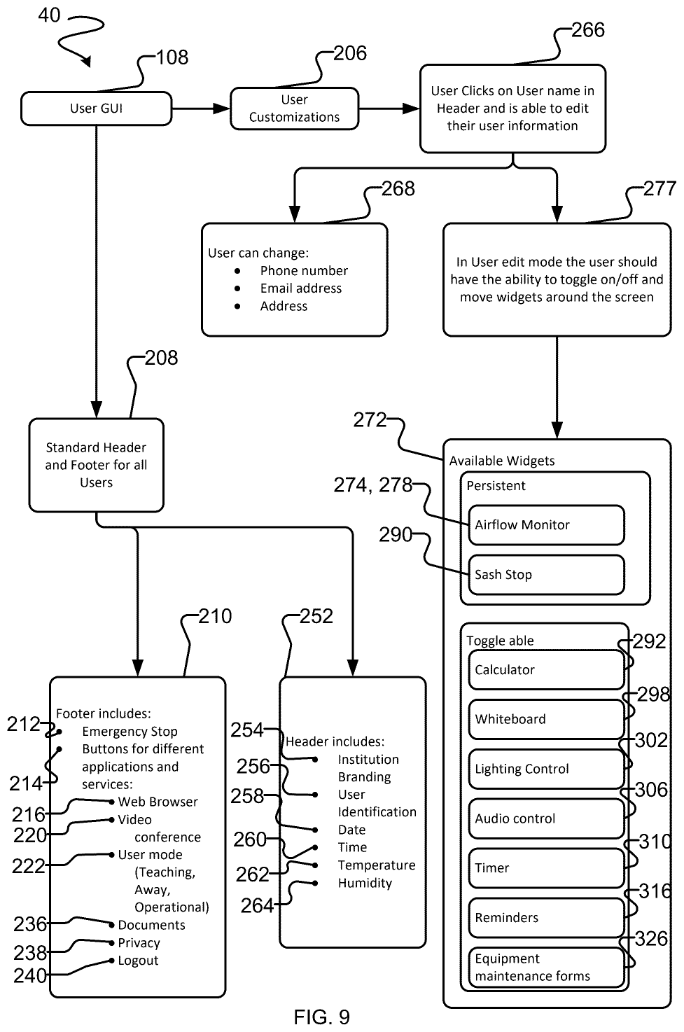

As seen in FIG. 9, fume hood assembly 40 may include a customizable said GUI 108. This may include user customizations 206. According to one non-limiting embodiment, there may be provided standard headers and footers for each user as shown by box 208. As shown by box 210, non-limiting indicia/features for the footers may include an emergency stop button 212 which is touchscreen engaged and which automatically closes sash 84 and thereafter actuates locking mechanism 100 to its locked position seen in FIG. 2.

GUI 108 may additionally include in this non-limiting embodiment various software applications accessible/set-out via icons/buttons 214 along the footer thereof. This includes a web browser 216 accessible via the GUI. As seen in FIG. 5, fume hood assembly 40 is operatively connected to a remote server in this non-limiting embodiment via a network, in this case the Internet via an Internet connectivity unit 218. The fume hood assembly may operatively connect to and/or function via a cloud computing system. As seen in FIG. 5, fume hood assembly 40 includes an output unit 79 via which processor 68 may operatively connect to a remote server and/or a cloud computing system. The fume hood assembly in this non-limiting example includes an input unit 81 operatively connected to the processor and via which files, documents or like may be uploaded and stored within memory module 82.

Referring back to FIG. 9, fume hood assembly 40 may include video conferencing application software 220 accessible via GUI 108. The GUI is thus operable in this non-limiting embodiment to initiate or receive video conference calling and may be a part of icons/buttons 214.

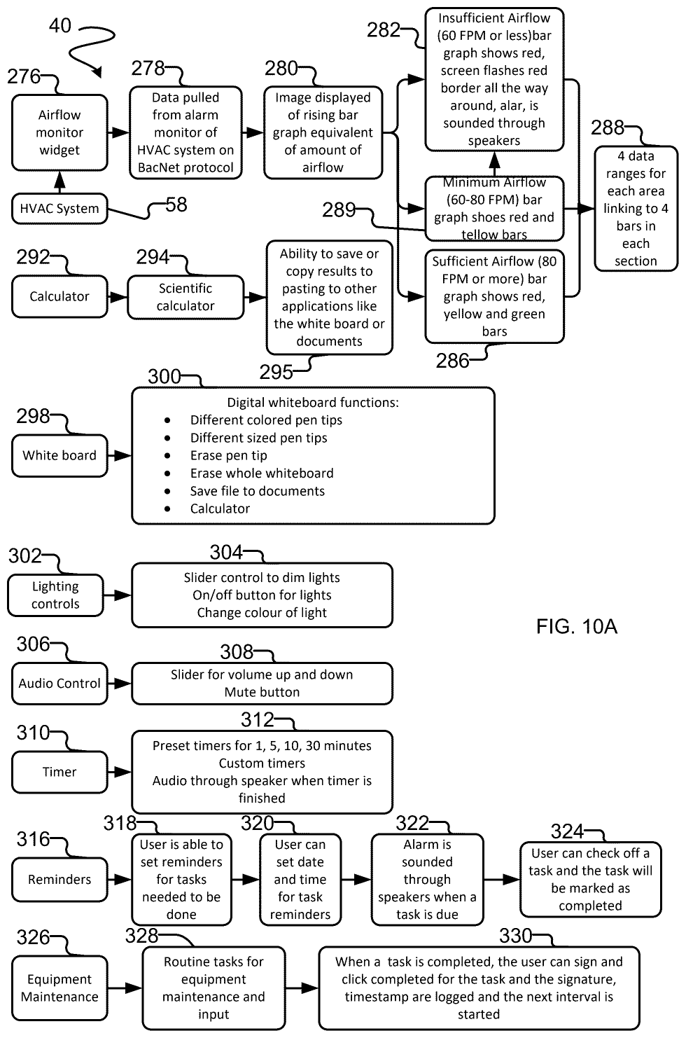

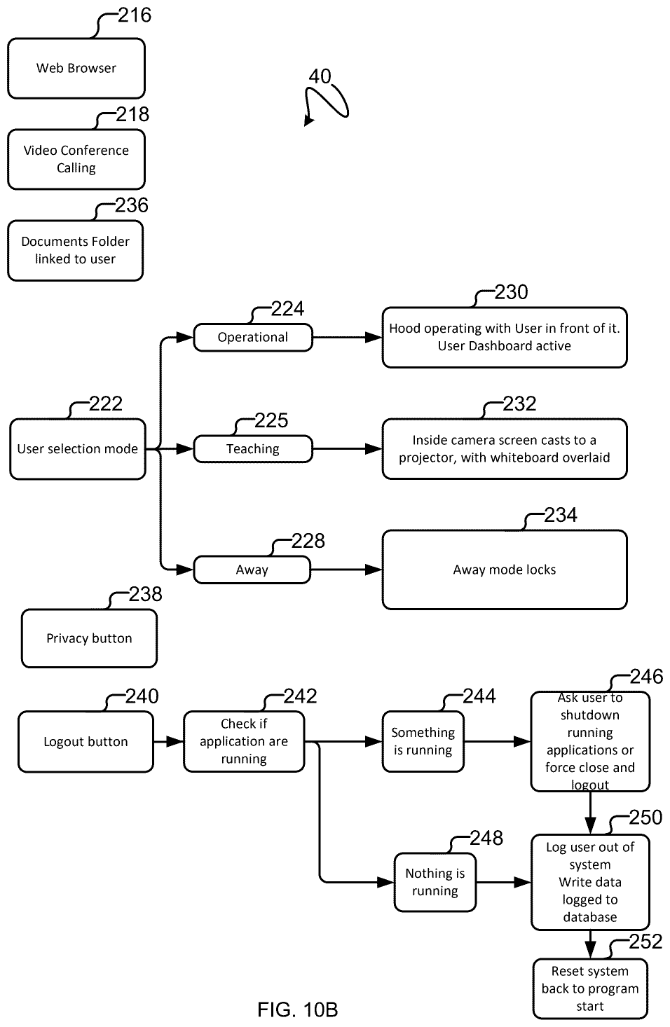

Still referring to FIG. 9, fume hood assembly 40 in this non-limiting embodiment includes a user mode selection feature or software application 222 accessible via GUI 108. The user mode software application may be a part of icons/buttons 214. As seen in FIG. 10B, user mode software application 222 in this non-limiting example includes i) an operational mode 224, ii) a teaching or remote-viewing mode 226 and iii) an away mode 228. For the operational mode and as seen by box 230, the fume hood operates with the user in front thereof and an enlarged set of features accessible upon the user successfully logging in. For the teaching or remote-viewing mode and as seen by box 232, video and/or images of interior of the fume hood as captured by the camera are displayed externally, such as to one or more students.

For the away mode and as seen by box 234, fume hood assembly 40 locks the sash thereof in the closed position, thereby inhibiting access to the interior of the fume hood. For the away mode, the fume hood assembly may continue to data log within a timer/folder associated with the user in this non-limiting embodiment. For the away mode, fume hood assembly enables a camera web server for the user to view the interior of the fume hood remotely via the Internet in this non-limiting example.

Referring back to FIG. 9, fume hood assembly 40 in this non-limiting embodiment includes a documents folder and/or software application 236. The documents software application is part of icons/buttons 214 in this non-limiting example. Documents software application 236 is operable via GUI 108 to create and edit documents which may be saved locally or remotely (e.g. Cloud-based) to a document management system. Fume hood assembly 40 may include a digital documents storage folder which is stored locally (within memory module 82 seen in FIG. 5) or remotely and linked to a specific user.

As seen in FIG. 9, fume hood assembly 40 may include a privacy mode, which may comprise an icon/button 238 of GUI 108 that is selectively actuated by the user. Display 106 inhibits viewing of interior 56 of fume hood 42 seen in FIG. 2 when in the privacy mode thereof. The display hides or conceals at least in part the interior of the fume hood when in the privacy mode thereof. Display 106 is opaque at least in part and in this example fully opaque in the privacy mode thereof according to one non-limiting example. The display is black in the privacy mode thereof according to another non-limiting embodiment. Display 106 thus enters the privacy mode thereof upon receiving one or more user-inputs according to one non-limiting embodiment.

The display as herein described is thus selectively at least partially transparent during operation thereof and the display hides or conceals at least in part an interior of the fume hood when in the privacy mode thereof. In addition or alternatively, the display in operational mode 230 thereof seen in FIG. 10B facilitates viewing therethrough into the interior of the fume hood. Fume hood assembly 40 in this non-limiting embodiment reverts back from the opaqueness of the display screen in the privacy mode, to the operational mode anew with the display being at least partially transparent by the user touching the screen once more. This may cause the screen to revert back to its dashboard setting again.

Processor 68 seen in FIG. 2 may in one non-limiting embodiment monitor user activity and cause display 106 to enter the privacy mode upon determining that a period of user inactivity exceeds a predetermined period of time or threshold. The display may thus in this non-limiting example enter a privacy mode within a predetermined period of time of inactivity thereon. A method of improving safety of fume hood assembly 40 may thus include rendering display 106 of sash 84 of fume hood 42 seen in FIG. 4 to be selectively opaque or transparent at least in part. The method may include enabling a first user to operate the fume hood while enabling a second user to monitor fume hood operation(s) using camera 76 situated within the fume hood.

A method of improving safety of fume hood assembly 40 may thus include configuring the display to be selectively or at least partially transparent. The method may include configuring display to be selectively at least partially transparent during operation thereof. The method may include configuring the display to hide or conceal at least in part interior of fume hood when in a privacy mode thereof.

Referring back to FIG. 9, fume hood assembly 40 includes a logout feature or button 240, which may be a part of buttons/icons 214. When the logout button is actuated and as seen in FIG. 10B, the fume hood assembly via the processor thereof next determines whether any applications or widgets are running as shown by box 242. If the processor determines that something is running as shown by box 244, then fume hood assembly 40 asks the user to shut down the running application/width or force close it and logout as shown by box 246. If the processor determines that nothing is running as shown by box 248, then the fume hood assembly logs the user out of the system and stores any data associated therewithin into its memory (and/or a database thereof) associated with that user as shown by box 250. Fume hood assembly 40 next resets its system back to program start or the black screen of the display while no user is present as shown by box 148 in FIG. 8.

Referring back to FIG. 9, GUI 108 may in this non-limiting embodiment include conveyed on a header 252 thereof institution branding 254, user identification 256, date 258, time 260, temperature data 262 and humidity data 264. Temperature and humidity sensors 70 and 72 seen in FIG. 1 output their readings/measurements visually in the form of the temperature and humidity data in this non-limiting example.

As seen in FIG. 9, GUI 108 in this non-limiting embodiment enables the user so authorized and successfully logged in to edit their user information as shown by box 266, in this non-limiting example by touching/clicking-on their user name in header 252. The user may thereafter change/update their contact information therewithin as shown by box 268. This may include phone number, email address and postal address information. In this edit mode, GUI 108 enables the user to toggle various features on or off, and move software applications and/or widgets around the screen as shown by box 270.

Fume hood assembly 40 provides a plurality of different modules or widgets and/or additional software applications accessible as generally shown by box 272 and which may be accessible upon access to the fume hood assembly being enabled. The application software and/or widgets are operatively connected to processor 68 and accessible via display 106 seen in FIG. 1.

For example and referring back to FIG. 10A, fume hood assembly 40 includes an airflow monitor module or widget 276. The airflow monitor widget pulls data from an alarm monitor of HVAC system 58 seen in FIG. 1 in one non-limiting embodiment as shown by box 278 in FIG. 10B. Airflow monitor widget 276 pulls data from the alarm monitor of the HVAC system via a building automation and control network (BACnet) protocol in this non-limiting example. The alarm monitor may be separate from or a part of alarm system 69 seen in FIG. 1. In addition or alternatively, airflow monitor widget 276 receives and pulls data from airflow sensor 74 of an airflow monitoring system 75 seen in FIG. 1.

Still referring to FIG. 1, processor 68 and speaker 71 are operatively connected to the airflow monitor widget 276 seen in FIG. 10A in this non-limiting example. Display 106 seen in FIG. 1 operatively connects to the airflow monitor widget, with airflow rates passing through fume hood 42 being displayable thereon. This is shown by box 280 in FIG. 10A where an image is displayed of a rising bar graph substantially equivalent and/or corresponding to the amount of airflow passing through the fume hood. Processor 68 seen in FIG. 1 may determine if the airflow rate meets a threshold minimum or sufficient flowrate and convey the same via display 106. The threshold minimum flowrate of gas through interior 56 of fume hood 42 may be 60 feet per minute (FPM) in one non-limiting embodiment.

Fume hood assembly 40 in this non-limiting embodiment conveys a visual indicator upon the assembly determining that airflow through the fume hood is below the minimum predetermined threshold. The visual indicator in this non-limiting embodiment is conveyed on display 106. The visual indicator in this non-limiting example comprises a red color that is flashing. The visual indicator may comprise red about a border 107 of display 106 seen in FIG. 4 and/or a band along a perimeter of the sash or display thereof as shown by box 282 in FIG. 10A. Fume hood assembly 40 flashes indicia via the display and sounds an alarm via the speaker upon determining via processor, alarm monitor or otherwise, that airflow through fume hood is below a minimum predetermined threshold or threshold minimum flowrate. Alarm system 69 seen in FIG. 1 may thus be said to include display 106. The visual indicator may also include a bar graph showing red bars according to this non-limiting example. Display 106 selectively conveys yet an additional visual safety warning, such as DANGER, ALARM and/or WARNING indicia 109 seen in FIG. 4 thereon upon determining that one or more safety thresholds have been exceeded.

If fume hood assembly 40 determines that a minimum or threshold minimum flowrate range has been met, the assembly provides another visual indicator. In this non-limiting example, the fume hood assembly displays yellow and/or red and yellow indicia on the display thereof upon determining that a minimum airflow through the fume hood has been reached in this non-limiting example as shown by box 284 seen in FIG. 10A. The minimum airflow may be between 60 and 80 feet per minute (FPM) in one non-limiting embodiment.

If fume hood assembly 40 determines that a more preferred or sufficient flowrate range has been met, the assembly provides yet another visual indicator. In this non-limiting example, the fume hood assembly displays green and/or red, yellow and green indicia on the display thereof in this non-limiting embodiment upon determining that a sufficient airflow through the fume hood has been reached as seen by box 286. The sufficient airflow may be an airflow that is equal to or greater than 80 feet per minute (FPM) in one non-limiting embodiment. The threshold sufficient flow rate may be between 70 and 90 feet per minute (FPM) in another non-limiting embodiment.

Fume hood assembly 40 in this non-limiting example includes four data ranges or bars of indicia indicative of the extent to which there is insufficient airflow, minimum airflow and sufficient airflow, respectively, as shown by box 288.

The method of improving safety of fume hood assembly 40 may thus include displaying an airflow rate on a fume hood sash. The displaying step may include indicating via the display if the airflow meets a threshold minimum flowrate. The displaying step may include indicating via the display if the airflow rate meets a threshold sufficient flowrate. The method may include triggering an alarm if the airflow rate is less than the minimum predetermined threshold. The triggering the alarm step may include sounding alarm through speaker connected to the fume hood. The triggering the alarm step may include flashing of visual indicator on the sash.

As seen in FIG. 9, the plurality of widgets 272 in this non-limiting example include a sash positioning or stop module or widget 290. The sash stop widget facilitates positioning of sash 84 seen in FIG. 1, in this non-limiting example inhibiting movement of the sash once a desired position thereof (e.g. fully open or fully closed) has been reached. Sash stop widget 290 seen in FIG. 9 may operatively connect to processor 68 and/or locking mechanism 100 seen in FIG. 1 to achieve this functionality at least in part.

Referring back to FIG. 9, fume hood assembly 40 in this non-limiting embodiment includes application software or a widget in the form a virtual calculator 292. As seen in FIG. 10A, the calculator may be a scientific calculator to calculate/process formulas as seen by box 294. Virtual calculator 292 may also have the ability to save or copy a result or output of a calculation, into one or more other applications of the fume hood assembly as shown by box 296, such as within documents software application 236 or the like seen in FIG. 9.

Still referring to FIG. 9, fume hood assembly 40 in this non-limiting embodiment includes a widget or application software which provides the functionality of a whiteboard in a digital environment, in this example digital whiteboard widget or application software 298. As seen in FIG. 4, display 106 enables notes indicia 110 to be created by selectively applying pressure thereon, such as via pen or stylus. The notes indicia may comprise user-inputted data and/or user-created information. The user-created information may in this non-limiting embodiment include one or more of an equation, an observation, a discovery, a memory cue and a safety warning. Display 106 conveys user-inputted data and/or indicia 110 thereon in real-time.

Processor 68 receives user-inputted data via the display and stores the same locally or remotely in memory or memory module 82 for selective retrieval at a later time. In this non-limiting embodiment and referring back to FIG. 4, the processor is operatively connected to display 106 so as to receive one or more signals relating to notes indicia 110. Processor 68 in this non-limiting example converts the one or more signals into text-based information to be stored locally for future retrieval and/or to be transmitted to a remote server. Display 106 as herein described may thus be said to comprise a digital whiteboard. Referring to FIG. 10B, for the teaching or remote-viewing mode as shown by box 232, video of the interior of the fume hood as captured by camera may be displayed externally to one or more students with an overlay of anything written on digital whiteboard. In addition or alternatively, virtual calculator 292 may enable copying and pasting therefrom to the digital whiteboard.

As seen in FIG. 10A, whiteboard application software 298 may provide a variety of functionality as set out in box 300. This may include one or more of: enabling or provide virtual pen colors and pen tip and pen eraser sizes to be customizable; selective erasing of the entire whiteboard; and/or enabling saving of files to a documents folder locally or remotely for future retrieval via whiteboard application software. A method of improving safety of fume hood assembly 40 may thus include configuring the display as a GUI with a medium via which notes indicia are created and/or stored.

Referring back to FIG. 9, fume hood assembly 40 may include a lighting widget or software application 302 operable/accessible via display 106 seen in FIG. 1. As shown in FIG. 10A and by box 304, the lighting software application enables lighting of the fume hood assembly or within a room thereof, to be controllable. Lighting software application 302 may enable the lighting to be selectively turned on or off and/or selectively dimmed and/or may enable the color of the lighting to be selectively changed or adjusted in non-limiting embodiments.

As seen in FIG. 9, fume hood assembly 40 may include an audio software application or widget 306 operable/accessible via display 106 seen in FIG. 1. As shown in FIG. 10A and by box 308, the audio widget enables audio volumes for speaker 71 of fume hood assembly 40 seen in FIG. 1, to be controllable: either raising or lowering the volume or muting sound, for example.

The above are non-limiting examples of applications, widgets and/or devices included as part of and/or connectable to fume hood assembly 40. For example, the fume hood assembly may additionally include coupled thereto additional applications, widgets and/or devices, such as Bluetooth (R) accessories, and/or couple to a keyword, mouse and the like.

Referring back to FIG. 9, fume hood assembly 40 may include a timer widget or software application 310 operable/accessible via display 106 seen in FIG. 1. As shown in FIG. 10A and by box 312, the timer software application may include one or more of: preset timers (e.g. for 1, 5, 10 and 30 minutes); a custom timer feature; and/or an audio output operatively connected to speaker 71 seen in FIG. 1 upon one or more said timers being completed.

As seen in FIG. 9, fume hood assembly 40 in this non-limiting embodiment includes a reminder system, widget and/or software application 314 operable/accessible via display 106 seen in FIG. 1. As shown in FIG. 10A and by box 316, the reminder software application in this example enables user-created reminders at specific dates and times for tasks to be completed. Reminder software application 318 enables the authorized/certified user to set date and time information for a task reminder as seen by box 320. The reminder software application outputs a notification when a given said task is due a shown by box 322. In this non-limiting example, reminder software application 316 operatively connects to speaker 71 seen in FIG. 1, with an audio notification being outputted via the speaker when a given said task is due for example. Referring back to FIG. 10A, the reminder software application may create a time-stamped/logged record when respective said tasks have been completed as shown by box 324.

The method of improving safety of fume hood assembly 40 may thus include setting reminders on display 106 coupled to sash 84 of fume hood 42 seen in FIG. 3. The method may include triggering an alarm when the reminder is due. The triggering the alarm step may include sounding an alarm through speaker 71 coupled to fume hood 42.

Referring back to FIG. 9, fume hood assembly 40 includes an equipment maintenance form(s), widget and/or software application 326. As shown in FIG. 10A and by box 328, the equipment maintenance software application may include one or more tasks related thereto. Equipment maintenance software application 326 enables routine tasks for equipment maintenance to be inputted, monitored, logged and/or tracked. When a given task thereof is completed, the equipment maintenance software application creates a timestamp thereof in this non-limiting embodiment as shown by box 330. Equipment maintenance software application 326 in this non-limiting embodiment requires a digital signature from a user in order to thereafter log and timestamp a given said task as complete. When a maintenance task is determined to be complete, the equipment maintenance software application in this non-limiting example automatically re-sets the deadline for completing the maintenance task anew at a future date and time once more.

A method of improving safety of fume hood assembly 40 may thus include displaying equipment maintenance information on display 106 coupled to sash 84 of fume hood 42 seen in FIG. 3. The method may include identifying users of the fume hood, logging user identification information corresponding to a user who performed a maintenance task, logging a date the maintenance task is performed, logging a time the maintenance task is performed, and displaying a reminder for the next maintenance task.

Display 106 as herein described may thus enable one or more of: selective monitoring and/or control of airflow of fume hood; calculations to be performed via a virtual calculator; digital notes to be taken via a digital whiteboard; selective adjustment of lighting controls for lighting within or near the fume hood or within a room thereof; selective adjustment of audio controls of one or more speakers thereof; selective operation of one or more digital timers; selectively operation of a digital reminder system; and selective monitoring and/or performance of equipment maintenance.

It will be appreciated that many variations are possible within the scope of the invention described herein.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, code for configuring a configurable logic circuit, applications, apps, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software and other modules may reside on servers, workstations, personal computers, tablet computers, and other devices suitable for the purposes described herein.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value ±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and.
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5; and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternatives or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

### Additional Description

Examples of fume hood assemblies have been described, as well as methods of improvement fume hood assembly comprising the same. The following clauses are offered as further description.

(1) A laboratory safety enclosure assembly comprising: a laboratory safety enclosure including a sash, window and/or door; and a user-identification system operatively connected to the laboratory safety enclosure such that operation thereof is a function of the user-identification system detecting a pre-authorized user.

(2) A laboratory safety enclosure assembly comprising: a laboratory safety enclosure including a sash, window and/or door; and a radio frequency identification (RFID) reader operatively connected to the laboratory safety enclosure such that operation thereof is a function of the RFID reader interacting with one or more RFID tags.

(3) A laboratory safety enclosure assembly comprising: a laboratory safety enclosure including a sash, window and/or door; and a sensor operatively connected to the laboratory safety enclosure such that operation thereof is a function of the sensor interacting with one or more objects.

(4) A laboratory safety enclosure assembly according to any clause herein, wherein the sensor comprises a radio frequency identification (RFID) reader and wherein the one or more objects comprise one or more RFID cards or tags.

(5) A laboratory safety enclosure assembly according to any clause herein, including a locking mechanism, with unlocking thereof being a function of the RFID reader interacting with one or more RFID tags.

(6) A laboratory safety enclosure assembly according to any clause herein, wherein the locking mechanism operatively connects to the RFID reader.

(7) A laboratory safety enclosure assembly comprising: a laboratory safety enclosure has an interior and including a sash, window and/or door; a locking mechanism coupled to the sash, window and/or door so as to inhibit access to the interior of the laboratory safety enclosure; and a user-identification system operatively connected to the locking mechanism, with unlocking thereof being a function of the user-identification system detecting a pre-authorized user.

(8) A laboratory safety enclosure assembly comprising: a laboratory safety enclosure having an interior and including a sash, window and/or door; a locking mechanism coupled to the sash, window and/or door so as to inhibit access to the interior of the laboratory safety enclosure; and a radio frequency identification (RFID) reader operatively connected to the locking mechanism, with unlocking thereof being a function of the RFID reader interacting with one or more RFID tags.

(9) A laboratory safety enclosure assembly comprising: a laboratory safety enclosure having an interior and including a sash, window and/or door; a locking mechanism coupled to the sash, window and/or door so as to inhibit access to the interior of the laboratory safety enclosure; and a sensor operatively connected to the locking mechanism, with unlocking thereof being a function of the sensor interacting with one or more objects.

(10) A laboratory safety enclosure assembly comprising: a laboratory safety enclosure including a sash, window and/or door; a display coupled to the sash, window and/or door; and a user-identification system operatively connected to the display such that operation thereof is a function of the user-identification system detecting a pre-authorized user.

(11) A laboratory safety enclosure assembly comprising: a laboratory safety enclosure including a sash, window and/or door; a display coupled to the sash, window and/or door; and a radio frequency identification (RFID) reader operatively connected to the display such that operation thereof is a function of the RFID reader interacting with one or more RFID tags.

(12) A laboratory safety enclosure assembly comprising: a laboratory safety enclosure including a sash, window and/or door; a display coupled to the sash, window and/or door; and a sensor operatively connected to the display such that operation thereof is a function of the sensor interacting with one or more objects.

(13) A laboratory safety enclosure assembly according to any clause herein, including a display coupled to the sash, window and/or door.

(14) A laboratory safety enclosure assembly according to any clause herein, wherein the sash, window and/or door comprises said display or vice versa.

(15) A laboratory safety enclosure assembly according to any clause herein, wherein the display operatively connects to the RFID reader.

(16) A laboratory safety enclosure assembly of any clause herein, wherein the one or more RFID tags are pre-authorized said RFID tags.

(17) A laboratory safety enclosure assembly of any clause herein, including a processor operatively connected to the RFID reader, with the processor being configured to determine whether said one or more RFID tags is authorized.

(18) A laboratory safety enclosure assembly of any clause herein, wherein the sash, window and/or door is positionable to inhibit access to the interior of the laboratory safety enclosure and wherein the processor is configured to selectively enable the sash, window and/or door to move so as to enable access to the interior of the laboratory safety enclosure upon one or more criteria being met.

(19) A laboratory safety enclosure assembly of any clause herein, wherein the processor is configured to selectively actuate the locking mechanism to an unlocked position upon determining that said one or more RFID tags is authorized.

(20) A laboratory safety enclosure assembly of any clause herein, wherein the processor is configured to enable operation of and/or actuate the laboratory safety enclosure upon determining that said one or more RFID tags is authorized.

(21) A laboratory safety enclosure assembly of any clause herein, wherein the processor is configured to enable operation of an expanded set of features of the display upon determining that said one or more RFID tags is authorized.

(22) A laboratory safety enclosure assembly according to any clause herein, wherein the RFID reader outputs a signal upon interacting with said one or more RFID tags, and wherein the assembly includes a processor configured to receive said signal and unlock or actuate from the locking mechanism based thereon.

(23) A laboratory safety enclosure assembly of any clause herein, including a processor operatively connected to the RFID reader, the display and/or the locking mechanism.

(24) A laboratory safety enclosure assembly of any clause herein, including an occupancy sensor or person-detecting device configured to detect the presence of a person near or adjacent the laboratory safety enclosure and emit a signal in response thereto.

(25) A laboratory safety enclosure assembly of any clause herein, wherein the person-detecting device is a sensor, a passive infrared sensor, a millimeter wave (mmWave) sensor, a camera sensor and/or combination thereof.

(26) A laboratory safety enclosure assembly according to any clause herein, including a sensor for detecting the presence of a user, the sensor operatively connecting to the laboratory safety enclosure.

(27) A laboratory safety enclosure assembly of any clause herein, wherein the person-detecting device is a camera.

(28) A laboratory safety enclosure assembly of any clause herein, wherein the processor is operatively connected to the person-detecting device.

(29) A laboratory safety enclosure assembly of any clause herein, including a processor which receives said signal from the person-detecting device and in response thereto operatively prompts the person to scan an RFID card or tag via the RFID reader.

(30) A laboratory safety enclosure assembly of any clause herein, wherein the RFID reader reads information contained within the RFID card or tag and communicates said information to the processor and wherein the processor determines whether to enable access to the laboratory safety enclosure based on said information.

(31) A laboratory safety enclosure assembly of any clause herein, wherein the RFID reader reads user information contained within the RFID card or tag and communicates said user information to the processor, wherein the processor determines whether said user information comprises a user who has successfully completed a certification process and if so, the processor causes access to the laboratory safety enclosure to be enabled.

(32) A laboratory safety enclosure assembly of any clause herein, wherein the processor is configured to enable access to the laboratory safety enclosure by actuating the locking mechanism to the unlocked position.

(33) A laboratory safety enclosure assembly of any clause herein, wherein the processor is configured to enable access to the laboratory safety enclosure by enabling an expanded set of features of the display to be accessible.

(34) A laboratory safety enclosure assembly according to any clause herein, including a memory module configured to operatively connect to the processor and selectively store information thereon.

(35) A laboratory safety enclosure assembly of any clause herein, including memory via which a database is stored and wherein the processor determines whether said user information is in the database and if no, the processor takes no action and/or inhibits access to the laboratory safety enclosure.

(36) A laboratory safety enclosure assembly of any clause herein, wherein if the processor both determines that said user information is in the database and determines that a user associated with said user information has successfully completed a safety training and/or certification process, then the processor enables access to the laboratory safety enclosure.

(37) A laboratory safety enclosure assembly of any clause herein, wherein if the processor determines that said user information is in the database but corresponds to a user who has not successfully completed a safety training and/or certification process, then the processor prompts the user to continue with training via the display.

(38) A laboratory safety enclosure assembly of any clause herein, wherein the training comprises training materials.

(39) A laboratory safety enclosure assembly of any clause herein, wherein the training comprises a safety training video shown via the display.

(40) A laboratory safety enclosure assembly of any clause herein, wherein the training comprises a quiz with satisfactory completion thereof requiring the user to meet or exceed a predetermined test score and/or threshold.

(41) A laboratory safety enclosure assembly of any clause herein, wherein the processor is configured to enable access to the laboratory safety enclosure to the user upon determining that the training has been completed and/or the user has completed the quiz with a test score that meets or exceeds the predetermined test score and/or threshold.

(42) A laboratory safety enclosure assembly comprising: a laboratory safety enclosure having an interior and including a sash, window and/or door; and a display coupled to the sash, window and/or door; and a processor and/or user-identification system configured to require viewing and/or completion of a safety training video or course via the display prior to enabling access to the interior of the laboratory safety enclosure.

(43) A laboratory safety enclosure assembly comprising: a laboratory safety enclosure having an interior and including a sash, window and/or door; and a display coupled to the sash, window and/or door; and a processor and/or user-identification system configured to restrict access to the interior of the laboratory safety enclosure to users who are pre-authorized and/or who have viewed and/or completed of a safety training video or course via the display.

(44) A laboratory safety enclosure assembly comprising: a laboratory safety enclosure having an interior and including a sash, window and/or door; and a display coupled to the sash, window and/or door; and a processor and/or user-identification system configured to require satisfactory completion of a safety test via the display prior to enabling access to the interior of the laboratory safety enclosure.

(45) A laboratory safety enclosure assembly comprising: a laboratory safety enclosure having an interior and including a sash, window and/or door; and a display coupled to the sash, window and/or door; and a processor and/or user-identification system configured to restrict access to the interior of the laboratory safety enclosure to users who are pre-authorized, who have viewed and/or completed of a safety training video or course via the display and/or who have completed a safety test via the display with a test score that equals to or exceeds a predetermined threshold.

(46) A laboratory safety enclosure assembly according to any clause herein, including a locking mechanism operatively connected to the sash, window and/or door and the processor and via which the processor selectively inhibits access to the interior of the laboratory safety enclosure.

(47) A laboratory safety enclosure assembly of any clause herein, wherein if the processor both determines that said user information is in the database and determines that a user associated with said user information has successfully completed a safety training and/or certification process, then the processor enables access to the laboratory safety enclosure.

(48) A laboratory safety enclosure assembly of any clause herein, wherein if the processor determines that said user information is in the database but corresponds to a user who has not successfully completed a safety training and/or certification process, then the processor prompts the user to continue with training via the display.

(49) A laboratory safety enclosure assembly of any clause herein, including a servomotor in communication with the processor and via which the sash, window and/or door is selectively unlocked.

(50) A laboratory safety enclosure assembly of any clause herein, wherein the display is configured to provide customized or customizable settings based on a given said user.

(51) A laboratory safety enclosure assembly comprising: a laboratory safety enclosure having an interior and including a sash, window and/or door; a locking mechanism coupled to the sash, window and/or door so as to inhibit access to the interior of the laboratory safety enclosure; and a display operatively connected to the locking mechanism, with the locking mechanism being selectively unlockable via the display.

(52) A laboratory safety enclosure assembly according to any clause herein, wherein the display is a graphical user interface.

(53) A laboratory safety enclosure assembly according to any clause herein, wherein the graphical user interface is a touchscreen.

(54) A laboratory safety enclosure assembly according to any clause herein, wherein the display is capable of being transparent at least in part.

(55) A laboratory safety enclosure assembly according to any clause herein, wherein the display is configured to be selectively transparent at least in part.

(56) A laboratory safety enclosure assembly comprising: a laboratory safety enclosure including a sash, window and/or door; and a display coupled to the sash, window and/or door, the display being selectively or at least partially transparent, and the display comprising a graphical user interface with a medium via and/or upon which notes indicia may be created.

(57) A laboratory safety enclosure assembly according to any clause herein, wherein the medium is a touchscreen.

(58) A laboratory safety enclosure assembly according to any clause herein, including a stylus via which said notes indicia are created on the said touchscreen.

(59) A laboratory safety enclosure assembly according to any clause herein, wherein said notes indicia comprises user-created information.

(60) A laboratory safety enclosure assembly according to any clause herein, wherein the user-created information comprises one or more of: an equation, an observation, a discovery, a memory cue and a safety warning.

(61) A laboratory safety enclosure assembly according to any clause herein, wherein the graphical user interface is configured to display experiment data thereon, including one or more information, status and/or indicia related thereto.

(62) A laboratory safety enclosure assembly according to any clause herein, wherein the processor is configured to receive user-inputted data via the display.

(63) A laboratory safety enclosure assembly according to any clause herein, wherein the display is configured to store and/or display user-inputted data and/or indicia thereon in real-time.

(64) A laboratory safety enclosure assembly according to any clause herein, wherein the processor is operatively connected to the display so as to receive one or more signals relating to said notes indicia, with the processor being configured to convert said one or more signals into text-based information to be stored locally for future retrieval and/or to be transmitted to a remote server.

(65) A laboratory safety enclosure assembly according to any clause herein, wherein the display comprises a digital whiteboard.

(66) A laboratory safety enclosure assembly comprising: a laboratory safety enclosure including a sash, window and/or door; and a display coupled to the sash, window and/or door, the display being selectively at least partially transparent and comprising a digital whiteboard.

(67) A laboratory safety enclosure assembly according to any clause herein, wherein the display comprises an application software which provides the functionality of a whiteboard in a digital environment.

(68) A laboratory safety enclosure assembly according to any clause herein, wherein the application software enables: virtual pen colors and pen tip and pen eraser sizes to be customizable; selective erasing of the entire whiteboard; and/or saving of files to a documents folder locally or remotely for future retrieval.

(69) A laboratory safety enclosure assembly according to any clause herein, wherein the display is operable to access a document management system.

(70) A laboratory safety enclosure assembly according to any clause herein, wherein the display is operable to create documents.

(71) A laboratory safety enclosure assembly according to any clause herein, wherein the display is operable to edit documents.

(72) A laboratory safety enclosure assembly according to any clause herein, wherein the display includes application software comprising a virtual calculator.

(73) A laboratory safety enclosure assembly according to any clause herein, wherein the display is configured to enable one or more of: selective monitoring and/or control of airflow of the laboratory safety enclosure; calculations to be performed via a virtual calculator; digital notes to be taken via a digital whiteboard; selective adjustment of lighting controls for lighting within or near the laboratory safety enclosure or within a room thereof; selective adjustment of audio controls of one or more speakers thereof; selective operation of one or more digital timers; selectively operation of a digital reminder system; and selective monitoring and/or performance of equipment maintenance.

(74) A laboratory safety enclosure assembly according to any clause herein, including an airflow monitoring system or widget.

(75) A laboratory safety enclosure assembly according to any clause herein, including an alarm.

(76) A laboratory safety enclosure assembly according to any clause herein, wherein the display is configured to operatively connect and/or be connectable to a heating, ventilation and air conditioning (HVAC) control system so as to display information therefrom thereon.

(77) A laboratory safety enclosure assembly according to any clause herein, wherein the laboratory safety enclosure assembly is configured to integrate with a heating, ventilation and air conditioning (HVAC) control system so as to pull/receive information/data therefrom.

(78) A laboratory safety enclosure assembly according to any clause herein, wherein the airflow monitoring system or widget is configured to pull data from an alarm monitor of a heating, ventilation and air conditioning (HVAC) system.

(79) A laboratory safety enclosure assembly according to any clause herein, wherein the airflow monitoring system or widget is configured to pull data from the alarm monitor of the HVAC system via a building automation and control network (BACnet) protocol.

(80) A laboratory safety enclosure assembly according to any clause herein, including a speaker.

(81) A laboratory safety enclosure assembly according to any clause herein, including a speaker operatively connected to the airflow monitoring system or widget, wherein the display operatively connects to the airflow monitoring system or widget and wherein the assembly is configured to flash indicia via the display and sound an alarm via the speaker upon determining that airflow through the laboratory safety enclosure is below a predetermined threshold.

(82) A laboratory safety enclosure assembly according to any clause herein, wherein the assembly is configured to display and/or flash red thereon and/or display red about a border thereof upon the assembly determining that airflow through the laboratory safety enclosure is below a predetermined threshold.

(83) A laboratory safety enclosure assembly according to any clause herein, wherein the assembly is configured to display yellow and/or red and yellow indicia on the display thereof upon determining that a minimum airflow through the laboratory safety enclosure has been reached.

(84) A laboratory safety enclosure assembly according to any clause herein, wherein the assembly is configured to display green and/or red, yellow and green indicia on the display thereof upon determining that a sufficient airflow through the laboratory safety enclosure has been reached.

(85) A laboratory safety enclosure assembly according to any clause herein, wherein the assembly is configured to include four data ranges or bars of indicia indicative of the extent to which there is insufficient airflow, minimum airflow and sufficient airflow, respectively.

(86) A laboratory safety enclosure assembly according to any clause herein, wherein the display is configured to selectively convey a WARNING, DANGER and/or ALARM indicia thereon upon determining that one or more safety thresholds have been exceeded.

(87) A laboratory safety enclosure assembly according to any clause herein, including a lighting software application operable via the display and via which lighting thereof or within a room thereof is controllable.

(88) A laboratory safety enclosure assembly according to any clause herein, wherein the lighting software application enables the lighting to be selectively turned on or off and/or selectively dimmed and/or enables the color of the lighting to be selectively changed or adjusted.

(89) A laboratory safety enclosure assembly according to any clause herein, including a speaker and a timer software application operatively connected thereto, with the timer software application including preset timers and a custom timer feature and being configured to output an alarm via the speaker upon one or more said timers being completed.

(90) A laboratory safety enclosure assembly according to any clause herein, including a reminder system and/or software application operatively connected thereto, with the reminder system and/or software application being configured to enable user-created reminders at specific dates and times for tasks to be completed, with the reminder software application being configured to output a notification when a given said task is due and with the reminder software application being configured to create a time-stamped/logged record when respective said tasks have been completed.

(91) A laboratory safety enclosure assembly according to any clause herein, including a speaker operatively connected to the reminder system and/or software application, with the reminder software application being configured to output an audio said notification via the speaker when the given said task is due.

(92) A laboratory safety enclosure assembly according to any clause herein, including an equipment maintenance software application wherein when a given task thereof is completed, the equipment maintenance software application is configured to create a timestamp thereof.

(93) A laboratory safety enclosure assembly according to any clause herein, wherein the equipment maintenance software application enables routine tasks for equipment maintenance to be inputted, monitored, logged and/or tracked.

(94) A laboratory safety enclosure assembly according to any clause herein, wherein the equipment maintenance software application is configured to require a digital signature from a user in order to thereafter log and timestamp a given said task as complete.

(95) A laboratory safety enclosure assembly according to any clause herein, wherein when a maintenance task is determined to be complete, the equipment maintenance software application is configured to automatically re-set the deadline for completing the maintenance task anew at a future date and time once more.

(96) A laboratory safety enclosure assembly according to any clause herein, including an Internet connectivity unit.

(97) A laboratory safety enclosure assembly according to any clause herein, wherein the display is operable to access the Internet.

(98) A laboratory safety enclosure assembly according to any clause herein, wherein the assembly is connectable to the Internet and includes a web browser accessible via the display.

(99) A laboratory safety enclosure assembly according to any clause herein, wherein the assembly is connectable to the Internet and includes a video conferencing application software accessible via the display.

(100) A laboratory safety enclosure assembly according to any clause herein, wherein the display is operable to initiate or receive video conference calling.

(101) A laboratory safety enclosure assembly according to any clause herein, wherein the assembly includes a digital documents storage folder which is stored locally or remotely and linked to a specific user.

(102) A laboratory safety enclosure assembly according to any clause herein, wherein the assembly is operatively connected to a remote server.

(103) A laboratory safety enclosure assembly according to any clause herein, wherein the assembly is operatively connected to and/or functions via a cloud computing system.

(104) A laboratory safety enclosure assembly according to any clause herein, wherein the assembly includes a user mode selection feature accessible via the display and comprising an operational mode, a teaching or remote-viewing mode and an away mode.

(105) A laboratory safety enclosure assembly according to any clause herein, wherein for the operational mode, the laboratory safety enclosure operates with the user in front thereof and an enlarged set of features are configured to be accessible upon the user successfully logging in.

(106) A laboratory safety enclosure assembly according to any clause herein, including a camera positioned within and/or configured to view the interior of the laboratory safety enclosure.

(107) A laboratory safety enclosure assembly according to any clause herein, wherein for the teaching or remote-viewing mode, video of the interior of the laboratory safety enclosure as captured by the camera is displayed externally to one or more students.

(108) A laboratory safety enclosure assembly according to any clause herein, wherein for the teaching or remote-viewing mode, video of the interior of the laboratory safety enclosure as captured by the camera is displayed externally to one or more students with an overlay of anything written on the digital whiteboard.

(109) A laboratory safety enclosure assembly according to any clause herein, wherein for the away mode, the assembly is configured to lock the sash, window and/or door in a closed position which inhibits access to the interior of the laboratory safety enclosure.

(110) A laboratory safety enclosure assembly according to any clause herein, wherein for the away mode, the assembly is configured to continue to data log within the timer associated with the user.

(111) A laboratory safety enclosure assembly according to any clause herein, wherein for the away mode, the assembly is configured to enable a camera web server for the user to view the interior of the laboratory safety enclosure remotely via the Internet.

(112) A laboratory safety enclosure assembly according to any clause herein, wherein the processor is configured to monitor user activity and cause the display to enter a privacy mode upon determining that a period of user inactivity exceeds a predetermined period of time or threshold.

(113) A laboratory safety enclosure assembly according to any clause herein, wherein the display is configured to enter a privacy mode within a predetermined period of time of inactivity thereon.

(114) A laboratory safety enclosure assembly according to any clause herein, wherein the display is opaque in the privacy mode thereof.

(115) A laboratory safety enclosure assembly according to any clause herein, wherein the display includes a privacy mode.

(116) A laboratory safety enclosure assembly according to any clause herein, wherein the display is configured to enter a privacy mode upon receiving one or more user-inputs.

(117) A laboratory safety enclosure assembly according to any clause herein, wherein the display is black in the privacy mode thereof.

(118) A laboratory safety enclosure assembly according to any clause herein, wherein the display is configured to inhibit viewing of the interior of the laboratory safety enclosure when in the privacy mode thereof.

(119) A laboratory safety enclosure assembly according to any clause herein, wherein the display is configured to hide or conceal at least in part the interior of the laboratory safety enclosure when in the privacy mode thereof.

(120) A laboratory safety enclosure assembly comprising: a laboratory safety enclosure having an interior and including a sash, window and/or door; and a display coupled to the sash, window and/or door, the display being selectively at least partially transparent during operation thereof and the display being configured to hide or conceal at least in part the interior of the laboratory safety enclosure when in a privacy mode thereof.

(121) A laboratory safety enclosure assembly comprising: a laboratory safety enclosure having an interior and including a sash, window and/or door; and a display coupled to the sash, window and/or door, wherein the display is configured to selectively enter an operational mode in which the interior of the laboratory safety enclosure is visible therethrough and wherein the display is configured to selectively enter a privacy mode in which the display inhibits viewing of the interior of the laboratory safety enclosure therethrough.

(122) A laboratory safety enclosure assembly according to any clause herein, wherein the display comprises an electronic device for the visual presentation of data and/or information.

(123) A laboratory safety enclosure assembly of any clause herein, wherein the sash, window and/or door is transparent and/or comprises a slidable window which is moveable from a closed position, in which access to the interior of the laboratory safety enclosure is inhibited, to an open position, in which access to the interior of the laboratory safety enclosure is promoted.

(124) A laboratory safety enclosure assembly comprising: a laboratory safety enclosure having an interior and including a sash, window and/or door; a position-sensing device configured measure positioning of the sash, window and/or door and emit one or more signals based thereon; a display via which access to the interior of the laboratory safety enclosure is enabled; and a processor operatively connected to the position-sensing device and the display, with the processor being configured to log each change in positioning of the sash, window and/or door and the processor being configured to log time-in and time-out information and/or the time at which a logging-in event occurs and the time at which a logging-off event occurs.

(125) A laboratory safety enclosure assembly according to any clause herein, wherein the logging-in event occurs via the display and wherein the logging-out event occurs via the display.

(126) A laboratory safety enclosure assembly comprising: a laboratory safety enclosure including a sash, window and/or door; a position-sensing device configured measure positioning of the sash, window and/or door and emit one or more signals based thereon; a display via which features of the assembly are accessible upon logging-in with user information; and a processor operatively connected to the position-sensing device and the display, with the processor being configured to log and/or obtain data relating to each change in positioning of the sash, window and/or door and correlate the data so logged with the user information so as to determine a user-specific efficiency information, a user-specific safety score and/or a user-specific efficiency score.

(127) A laboratory safety enclosure assembly comprising: a laboratory safety enclosure having an interior and including a sash, window and/or door; a user-identification system configured to detect a pre-authorized user; a display coupled to the sash, window and/or door, the display including a digital whiteboard to record/store notes indicia thereon, the display being configured to be selectively transparent during operation thereof and to be opaque to inhibit viewing therethrough in a privacy mode; a locking mechanism coupled to the sash, window and/or door and which is selectively unlockable via the display; and a processor operatively connected to the user-identification system, the display and the locking mechanism, the processor being configured to restrict access to the interior of the laboratory safety enclosure to users who are pre-authorized, who have viewed and/or completed of a safety training video or course via the display and/or who have completed a safety test via the display with a test score that equals to or exceeds a predetermined threshold, the processor being configured to log and/or time-stamp changing in positioning of the sash, window and/or door and determine a user-specific safety score and/or a user-specific efficiency score therefrom.

(128) A laboratory safety enclosure assembly comprising: a laboratory safety enclosure having an interior and including a sash, window and/or door; a radio frequency identification (RFID) reader that interacts with one or more RFID tags to enable access to the interior of the laboratory safety enclosure; a display coupled to the sash, window and/or door, the display including a digital whiteboard to record/store notes indicia thereon, the display being configured to be selectively transparent during operation thereof and to be opaque to inhibit viewing therethrough in a privacy mode; a locking mechanism coupled to the sash, window and/or door and which is selectively unlockable via the display; and a processor configured to restrict access to the interior of the laboratory safety enclosure to users who are pre-authorized, who have viewed and/or completed of a safety training video or course via the display and/or who have completed a safety test via the display with a test score that equals to or exceeds a predetermined threshold, the processor being configured to log and/or time-stamp changing in positioning of the sash, window and/or door and determine a user-specific safety score and/or a user-specific efficiency score therefrom.

(129) A laboratory safety enclosure assembly comprising: a laboratory safety enclosure having an interior and including a sash, window and/or door; a sensor that interacts with one or more objects to enable access to the interior of the laboratory safety enclosure; a display coupled to the sash, window and/or door, the display including a digital whiteboard to record/store notes indicia thereon, the display being configured to be selectively transparent during operation thereof and to be opaque to inhibit viewing therethrough in a privacy mode; a locking mechanism coupled to the sash, window and/or door and which is selectively unlockable via the display; and a processor configured to restrict access to the interior of the laboratory safety enclosure to users who are pre-authorized, who have viewed and/or completed of a safety training video or course via the display and/or who have completed a safety test via the display with a test score that equals to or exceeds a predetermined threshold, the processor being configured to log and/or time-stamp changing in positioning of the sash, window and/or door and determine a user-specific safety score and/or a user-specific efficiency score therefrom.

(130) A laboratory safety enclosure assembly according to any clause herein, wherein the laboratory safety enclosure assembly comprises a fume hood said assembly.

(131) A laboratory safety enclosure assembly according to any clause herein, wherein the laboratory safety enclosure comprises a fume hood.

(132) A laboratory safety enclosure assembly according to any clause herein, wherein the laboratory safety enclosure assembly comprises a biosafety cabinet said assembly.

(133) A laboratory safety enclosure assembly according to any clause herein, wherein the laboratory safety enclosure comprises a biosafety cabinet.

(134) A sash, window and/or door for a laboratory safety enclosure, the sash, window and/or door comprising a display according to any clause therein.

(135) A display for a sash, window and/or door of a laboratory safety enclosure, the display being according to any clause therein.

(136) A kit for retrofitting a laboratory safety enclosure, the kit comprising: a user-identification system operatively connectable to the laboratory safety enclosure such that operation thereof is a function of the user-identification system detecting a pre-authorized user.

(137) A kit for retrofitting a laboratory safety enclosure to obtain a laboratory safety enclosure assembly according to any clause herein, the kit comprising a user-identification system operatively connectable to the laboratory safety enclosure such that operation thereof is a function of the user-identification system detecting a pre-authorized user.

(138) A kit for retrofitting a laboratory safety enclosure, the kit comprising a radio frequency identification (RFID) reader operatively connectable to the laboratory safety enclosure such that operation thereof is a function of the RFID reader interacting with one or more RFID tags.

(139) A kit for retrofitting a laboratory safety enclosure to obtain a laboratory safety enclosure assembly according to any clause herein, the kit comprising a radio frequency identification (RFID) reader operatively connectable to the laboratory safety enclosure such that operation thereof is a function of the RFID reader interacting with one or more RFID tags.

(140) A kit according to any clause herein, including a locking mechanism operatively connectable to a sash, window and/or door of the laboratory safety enclosure so as to inhibit access to an interior of the laboratory safety enclosure, wherein the user-identification system is operatively connected to the locking mechanism, with unlocking thereof being a function of the user-identification system detecting the pre-authorized user.

(141) A kit according to any clause herein, including a display operatively connectable to the sash, window and/or door, wherein the user-identification system is operatively connected to the display such that operation thereof is a function of the user-identification system detecting the pre-authorized user.

(142) A kit for retrofitting a laboratory safety enclosure to obtain a laboratory safety enclosure assembly according to any clause herein, including a position-sensing device connectable to the laboratory safety enclosure so as to measure positioning of the sash, window and/or door in real-time and emit one or more signals based thereon are emitted.

(143) A kit according to any clause herein, including a position-sensing device connectable to the laboratory safety enclosure so as to measure positioning of the sash, window and/or door in real-time and emit one or more signals based thereon are emitted.

(144) A kit for retrofitting a laboratory safety enclosure to obtain a laboratory safety enclosure assembly according to any clause herein, the kit comprising a display connectable to the laboratory safety enclosure, the display being operatively connectable to a servomotor of the sash, window and/or door to enable access to the interior of the laboratory safety enclosure.

(145) A kit according to any clause herein, including a display connectable to the laboratory safety enclosure, the display being operatively connectable to a servomotor of the sash, window and/or door to enable access to the interior of the laboratory safety enclosure.

(146) A kit for retrofitting a laboratory safety enclosure to obtain a laboratory safety enclosure assembly according to any clause herein, the kit comprising a display connectable to the laboratory safety enclosure, the display being operatively connectable to a locking mechanism of the sash, window and/or door to enable access to the interior of the laboratory safety enclosure; and

(147) A kit according to any clause herein, including a display connectable to the laboratory safety enclosure, the display being operatively connectable to a locking mechanism of the sash, window and/or door to enable access to the interior of the laboratory safety enclosure; and

(148) A kit according to any clause herein, including a processor operatively connected to the position-sensing device and the display, with the processor logging each change in positioning of the sash, window and/or door and the processor logging time-in and time-out information and/or the time at which a logging-in event occurs and the time at which a logging-off event occurs.

(149) A method of improving laboratory enclosure safety, the method comprising: coupling a user-identification system to a laboratory safety enclosure such that operation thereof is a function of the user-identification system detecting a pre-authorized user.

(150) A method of improving laboratory enclosure safety, the method comprising: coupling a radio frequency identification (RFID) reader to a laboratory safety enclosure such that operation thereof is a function of the RFID reader interacting with one or more RFID tags.

(151) A method of improving laboratory enclosure safety, the method comprising: coupling a locking mechanism to a sash, window and/or door of a laboratory safety enclosure so as to inhibit access to an interior of the laboratory safety enclosure; and operatively connecting a user-identification system to the locking mechanism, with unlocking thereof being a function of the user-identification system detecting a pre-authorized user.

(152) A method of improving laboratory enclosure safety, the method comprising: coupling a locking mechanism to a sash, window and/or door of a laboratory safety enclosure so as to inhibit access to an interior of the laboratory safety enclosure; and operatively connecting a radio frequency identification (RFID) reader to the locking mechanism, with unlocking thereof being a function of the RFID reader interacting with one or more RFID tags.

(153) A method of improving laboratory enclosure safety, the method comprising: coupling a display to a sash, window and/or door of a laboratory safety enclosure; and operatively connecting a user-identification system to the display such that operation thereof is a function of the user-identification system detecting a pre-authorized user.

(154) A method of improving laboratory enclosure safety, the method comprising: coupling a display to a sash, window and/or door of a laboratory safety enclosure; and operatively connecting a radio frequency identification (RFID) reader to the display such that operation thereof is a function of the RFID reader interacting with one or more RFID tags.

(155) A method of improving laboratory enclosure safety, the method comprising: coupling a display to a sash, window and/or door of a laboratory safety enclosure; and configuring the display to require viewing thereon and/or completion of a safety training video or course prior to enabling access to an interior of the laboratory safety enclosure.

(156) A method of improving laboratory enclosure safety, the method comprising: coupling a display to a sash, window and/or door of a laboratory safety enclosure; and restricting access to an interior of the laboratory safety enclosure to users who are pre-authorized and/or who have viewed and/or completed of a safety training video or course via the display.

(157) A method of improving laboratory enclosure safety, the method comprising: coupling a display to a sash, window and/or door of a laboratory safety enclosure; and configuring the display to require satisfactory completion of a safety test via the display prior to enabling access to an interior of the laboratory safety enclosure.

(158) A method of improving laboratory enclosure safety, the method comprising: coupling a display to a sash, window and/or door of a laboratory safety enclosure; and restricting access to an interior of the laboratory safety enclosure to users who are pre-authorized, who have viewed and/or completed of a safety training video or course via the display and who have completed a safety test via the display with a test score that equals to or exceeds a predetermined threshold.

(159) A method according to any clause herein, wherein the restricting step including coupling a locking mechanism to the sash, window and/or door which promotes positioning of the sash, window and/or door in a closed position.

(160) A method according to any clause herein, wherein the restricting step including selectively unlocking the locking mechanism via a processor to enable or facilitate access to the interior of the laboratory enclosure.

(161) A method of improving laboratory enclosure safety, the method comprising: coupling a locking mechanism to a sash, window and/or door of a laboratory safety enclosure so as to inhibit access to an interior of the laboratory safety enclosure; and operatively connecting a display to the locking mechanism, with the locking mechanism being selectively unlockable via the display.

(162) A method of improving laboratory enclosure safety, the method comprising: coupling a display to a sash, window and/or door of a laboratory safety enclosure; configuring the display to be selectively or at least partially transparent; and configuring the display as a graphical user interface with a medium via and/or upon which notes indicia are created and/or stored.

(163) A method of improving laboratory enclosure safety, the method comprising: coupling a display to a sash, window and/or door of a laboratory safety enclosure; configuring the display to be selectively at least partially transparent during operation thereof; and configuring the display to hide or conceal at least in part an interior of the laboratory safety enclosure when in a privacy mode thereof.

(164) A method of improving laboratory enclosure safety, the method comprising: monitoring positioning of a sash, window and/or door of a laboratory safety enclosure via a position-sensing device and emitting one or more signals based thereon; and configuring a processor to receive said one or more signals and log each change in positioning of the sash, window and/or door base on thereon.

(165) A method according to any clause herein, including providing a display via which features of the assembly are accessible upon logging-in with user information.

(166) A method according to any clause herein, including correlating via the processor the data so logged with the user information so as to determine user-specific safety information, a user-specific safety score, user-specific efficiency information and/or a user-specific efficiency score.

(167) A method for improving laboratory enclosure safety comprising: detecting whether a user is proximate to a laboratory safety enclosure; automatically locking a sash, window and/or door of the laboratory safety enclosure if the user is not proximate to the laboratory safety enclosure; and unlocking the sash, window and/or door when a safety criteria is satisfied.

(168) A method according to any clause herein, including within the detecting step, coupling an occupancy sensor or person-detecting device near or adjacent the laboratory safety enclosure, the person-detecting device being configured to detect the presence of the user and emit a signal in response thereto.

(169) A method according to any clause herein, including within the detecting step, determining via a processor that the user is proximate to the laboratory safety enclosure when the signal is equal to or exceeds a predetermined threshold.

(170) A method according to any clause herein, wherein the person-detecting device is a sensor, a passive infrared sensor, a millimeter wave (mmWave) sensor, a camera sensor and/or combination thereof.

(171) A method according to any clause herein, wherein the person-detecting device is a camera.

(172) A method according to any clause herein, including a step of inhibiting viewing through the sash, window and/or door of the laboratory safety enclosure when the user is not proximate to the laboratory safety enclosure.

(173) A method according to any clause herein, including a step of rendering the display of the laboratory safety enclosure opaque when the user is not proximate to the laboratory safety enclosure.

(174) A method according to any clause herein, including a step of rendering the laboratory safety enclosure transparent at least in part when the safety criteria is satisfied.

(175) A method according to any clause herein, wherein the safety criteria comprises answering a safety quiz displayed on the sash, window and/or door or on a display coupled to a front of the sash, window and/or door.

(176) A method according to any clause herein, wherein the safety criteria comprises displaying a video to the user.

(177) A method according to any clause herein, including as part of the safety criteria: determining via a processor whether a specific user is within a database of authorized said users.

(178) A method according to any clause herein, wherein identifying the specific user comprises scanning a radio frequency identification (RFID) tag.

(179) A method according to any clause herein, including identifying the specific user using a camera and facial-recognition software and/or technology

(180) A method according to any clause herein, displaying the video on the sash, window and/or door or on a display coupled to a front of the sash, window and/or door.

(181) A method for improving laboratory enclosure safety comprising displaying an airflow rate on a sash, window and/or door of a laboratory safety enclosure.

(182) A method according to any clause herein, wherein displaying an airflow rate further comprises indicating if the airflow rate meets a threshold sufficient flowrate.

(183) A method according to any clause herein, wherein the threshold sufficient flow rate is between 70 and 90 feet per minute (FPM).

(184) A method according to any clause herein, wherein the threshold sufficient flow rate is 80 feet per minute (FPM).

(185) A method according to any clause herein, wherein displaying an airflow rate further comprises indicating if the airflow meets a threshold minimum flowrate.

(186) A method according to any clause herein, wherein the threshold minimum flowrate is between 60 and 80 feet per minute (FPM).

(187) A method according to any clause herein, wherein the threshold minimum flowrate is 60 feet per minute (FPM).

(188) A method according to any clause herein, including triggering an alarm if the airflow rate is less than a minimum predetermined threshold.

(189) A method according to any clause herein, wherein triggering the alarm comprises sounding an alarm through speakers connected to the laboratory safety enclosure.

(190) A method according to any clause herein, wherein triggering the alarm comprises a flashing of a visual indicator on the sash, window and/or door.

(191) A method according to any clause herein, wherein the visual indicator is a band along a perimeter of the sash, window and/or door.

(192) A method according to any clause herein, wherein the visual indicator is red.

(193) A method for improving laboratory enclosure safety comprising setting reminders on a graphical user interface on a sash, window and/or door of a laboratory safety enclosure.

(194) A method according to any clause herein, further comprising triggering an alarm when the reminder is due.

(195) A method according to any clause herein, wherein the graphical user interface is a touchscreen.

(196) A method according to any clause herein, wherein the graphical user interface is at least partially transparent.

(197) A method according to any clause herein, wherein triggering the alarm comprises sounding an alarm through speakers connected to the laboratory safety enclosure.

(198) A method for improving laboratory enclosure safety comprising displaying equipment maintenance information on a graphical user interface on a sash, window and/or door of a laboratory safety enclosure.

(199) A method according to any clause herein, including comprising: identifying users of the laboratory safety enclosure; logging user identification information corresponding to a user who performed a maintenance task; logging a date the maintenance task is performed; logging a time the maintenance task is performed; and displaying a reminder for the next maintenance task.

(200) A method of improving laboratory enclosure security according to any clause herein, including enabling a sash, window and/or door of the laboratory safety enclosure to be change from at least partially transparent to opaque.

(201) A method according to any clause herein, wherein a sash, window and/or door of the laboratory safety enclosure is a graphical user interface.

(202) A method according to any clause herein, wherein the graphical user interface may be transparent or opaque as controlled by a user.

(203) A method for improving laboratory enclosure safety comprising enabling a first user to operate a laboratory safety enclosure while enabling a second user to monitor laboratory enclosure operation via a camera situated within the laboratory safety enclosure.

(204) A method of determining a rental rate for a laboratory safety enclosure assembly, the method comprising: configuring a position-sensing device to measure positioning of a sash, window and/or door of the laboratory safety enclosure assembly in real-time and emit one or more signals based thereon; logging via the position-sensing device each change in positioning of the sash, window and/or door and/or obtaining data via the position sensing device related thereto; correlating via a processor the data so logged with user information; and determining the rental rate of the laboratory safety enclosure assembly as a function of and/or proportionate to said data so correlated.

(205) A method of determining a rental rate for a laboratory safety enclosure assembly, the method comprising: configuring a position-sensing device to measure positioning of a sash, window and/or door of the laboratory safety enclosure assembly in real-time and emit one or more signals based thereon; logging via the position-sensing device each change in positioning of the sash, window and/or door and/or obtaining data via the position sensing device related thereto; correlating via a processor the data so logged with user information so as to determine user-specific safety information, a user-specific safety score, user-specific efficiency information and/or a user-specific efficiency score; and determining the rental rate of the laboratory safety enclosure assembly as a function of and/or proportionate to said user-specific safety information, user-specific safety score, user-specific efficiency information and/or user-specific efficiency score.

(206) A method according to any clause herein, including: tracking said user information by requiring a user to login to a display in order to have access to the laboratory safety enclosure assembly.

(207) A method according to any clause herein, including: logging alarm actuation information and correlating the same with said user information to determine the user-specific safety information, user-specific safety score, user-specific efficiency information and/or a user-specific efficiency score.

(208) A method according to any clause herein, including: monitoring the extent to which one or more alarms are triggered while the user is logged in and/or operating the laboratory safety enclosure assembly and increasing or discounting the rental rate based on thereon.

(209) A method according to any clause herein, wherein positioning of the sash, window and/or door for a period of time which exceeds a predetermined time threshold results in an increased rental rate.

(210) A method according to any clause herein, wherein positioning of the sash, window and/or door for a period of time which is less than said predetermined time threshold results in a discounted rental rate.

(211) A method according to any clause herein, including adjusting the rental rate of the laboratory safety enclosure assembly so determined based on user-specific safety information and/or a user-specific safety score.

(212) A method according to any clause herein, including: adjusting the rental rate of the laboratory safety enclosure assembly so determined based on the extent to which one or more alarms are actuated while the user is logged in and/or operating the laboratory safety enclosure assembly.

(213) A method according to any clause herein, wherein each log step includes logging in real-time and/or time-stamping.

(214) A method of improving safety of a laboratory safety enclosure assembly, the method comprising: requiring that a person be detected by the laboratory safety enclosure assembly via a person-detecting device as a first condition precedent and/or first safety layer/barrier for obtaining access to the laboratory safety enclosure assembly; requiring that the person be authorized and/or within a database in a memory of the laboratory safety enclosure assembly as a second condition precedent and/or second safety layer/barrier for obtaining access to the laboratory safety enclosure assembly; and requiring that the person be certified as a third condition precedent and/or third safety layer/barrier for obtaining access to the laboratory safety enclosure assembly.

(215) A method of any clause herein, including requiring that the person view training materials via a display of the laboratory safety enclosure assembly as part of the certification process as a fourth condition precedent and/or fourth safety layer/barrier for obtaining access to the laboratory safety enclosure assembly.

(216) A method of any clause herein, including requiring that the person satisfactorily pass a test or quiz via the display of the laboratory safety enclosure assembly as part of the certification process as a fifth condition precedent and/or fifth safety layer/barrier for obtaining access to the laboratory safety enclosure assembly.

(217) A method of improving safety of a laboratory safety enclosure assembly, the method comprising: requiring that a person be authorized and/or within a database in a memory of the laboratory safety enclosure assembly as a first condition precedent for obtaining access to the laboratory safety enclosure assembly; requiring that that the person be certified as a second condition precedent for obtaining access to the laboratory safety enclosure assembly; and if the person is not certified, requiring that that the person both i) view training materials via a display of the laboratory safety enclosure assembly as part of a certification process and ii) satisfactorily pass a test or quiz via the display of the laboratory safety enclosure assembly as part of the certification process, to obtain access to the laboratory safety enclosure assembly.

(218) A method according to any clause herein, wherein the laboratory safety enclosure assembly comprises a fume hood said assembly.

(219) A method according to any clause herein, wherein the laboratory safety enclosure comprises a fume hood.

(220) A method according to any clause herein, wherein the laboratory safety enclosure assembly comprises a biosafety cabinet said assembly.

(221) A method according to any clause herein, wherein the laboratory safety enclosure comprises a biosafety cabinet.

(222) A laboratory safety enclosure assembly comprising: a laboratory safety enclosure; a person-detecting device coupled to the laboratory safety enclosure; a memory; a processor operatively connected to the person-detecting device and the memory, the processor being configured to restrict access thereto unless i) a person is detected via the person-detecting device; ii) the person is authorized and/or within a database in said memory; and iii) the person is certified to use the laboratory safety enclosure assembly.

(223) A laboratory safety enclosure assembly according to any clause herein, wherein the processor determines that the person is certified based on the database in the memory of the laboratory safety enclosure assembly.

(224) A laboratory safety enclosure assembly comprising: a display; a memory; a processor operatively connected to the display and the memory, the processor being configured to restrict access thereto unless a person is authorized and certified to use the laboratory safety enclosure assembly as indicating within a database in said memory, whereby if the person as determined by the processor is not authorized and/or certified, the processor is configured to require that that the person both i) view training materials via the display of the laboratory safety enclosure assembly as part of a certification process and ii) satisfactorily pass a test or quiz via the display of the laboratory safety enclosure assembly as part of the certification process, to obtain access to the laboratory safety enclosure assembly.

(225) A laboratory safety enclosure assembly or method according to any clause herein, wherein the user-identification system comprises a fingerprint sensor, scanner and/or detection device.

(226) A laboratory safety enclosure assembly or method according to any clause herein, wherein the user-identification system comprises an iris sensor, scanner and/or detection device.

(227) A laboratory safety enclosure assembly or method according to any clause herein, wherein the user-identification system comprises a face sensor, scanner and/or detection device.

(228) A laboratory safety enclosure assembly or method according to any clause herein, wherein the user-identification system comprises a camera with artificial intelligence and/or facial recognition technology/software.

(229) A computer program product comprising a medium carrying computer readable instructions which, when executed by a processor, cause the processor to execute a process or method according to any clause herein.

(230) A server comprising a computer program product according to any clause herein, the server being configured to deliver the computer readable instructions to a recipient computer by a way of a communication medium.

(231) Apparatus including any new and inventive feature, combination of features, or sub-combination of features as described herein.

(232) Methods including any new and inventive steps, acts, combination of steps and/or acts or sub-combination of steps and/or acts as described herein.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A laboratory safety enclosure assembly comprising:
a laboratory safety enclosure including a sash, window and/or door; and
a user-identification system operatively connected to the laboratory safety enclosure such that operation thereof is a function of the user-identification system detecting a pre-authorized user.

2. A laboratory safety enclosure assembly as claimed in claim 1, wherein the user-identification system includes a radio frequency identification (RFID) reader operatively connected to the laboratory safety enclosure such that operation thereof is a function of the RFID reader interacting with one or more RFID tags.

3. A laboratory safety enclosure assembly as claimed in claim 1, including a locking mechanism coupled to the sash, window and/or door so as to inhibit access to an interior of the laboratory safety enclosure, wherein the user-identification system is operatively connected to the locking mechanism, with unlocking thereof being a function of the user-identification system detecting the pre-authorized user.

4. A laboratory safety enclosure assembly as claimed in claim 1, including a display coupled to the sash, window and/or door, wherein the user-identification system is operatively connected to the display such that operation thereof is a function of the user-identification system detecting the pre-authorized user.

5. A laboratory safety enclosure assembly according to claim 1, wherein the user-identification system comprises a biometric device comprising a fingerprint sensor, an iris sensor, and/or a face sensor, and/or wherein the user-identification system comprises a camera with artificial intelligence and/or facial recognition technology/software.

6. A laboratory safety enclosure assembly according to claim 1, including:
a person-detecting device coupled to the laboratory safety enclosure;
a memory;
a processor operatively connected to the person-detecting device and memory, the processor functioning to restrict access to the laboratory safety enclosure assembly unless
i) a person is detected via the person-detecting device;
ii) the person is authorized and/or within a database in said memory; and
iii) the person is certified to use the laboratory safety enclosure assembly.

7. A kit for retrofitting a laboratory safety enclosure to obtain the laboratory safety enclosure assembly of claim 1, the kit comprising one or more of:
a radio frequency identification (RFID) reader operatively connectable to the laboratory safety enclosure such that operation thereof is a function of the RFID reader interacting with one or more RFID tags;
a locking mechanism operatively connectable to the sash, window and/or door so as to inhibit access to an interior of the laboratory safety enclosure, wherein the user-identification system is operatively connected to the locking mechanism, with unlocking thereof being a function of the user-identification system detecting the pre-authorized user; or
a display operatively connectable to the sash, window and/or door, wherein the user-identification system is operatively connected to the display such that operation thereof is a function of the user-identification system detecting the pre-authorized user.

8. A laboratory safety enclosure assembly comprising:
a laboratory safety enclosure having an interior and including a sash, window and/or door; and
a display coupled to the sash, window and/or door; and
a processor and/or user identification system which requires viewing and/or completion of a safety training video or course via the display, to enable access to the interior of the laboratory safety enclosure.

9. A laboratory safety enclosure assembly according to claim 8, wherein the processor and/or user-identification system additionally requires satisfactory completion of a safety test via the display, so as to enable access to the interior of the laboratory safety enclosure.

10. A laboratory safety enclosure assembly comprising:
a laboratory safety enclosure including a sash, window and/or door; and
a display coupled to the sash, window and/or door, the display being selectively or at least partially transparent, and the display comprising a graphical user interface with a medium via which notes indicia may be created.

11. A laboratory safety enclosure assembly according to claim 10, wherein the display comprises a digital whiteboard.

12. A laboratory safety enclosure assembly according to claim 10, wherein the display is at least partially transparent during operation thereof and wherein the display hides or conceals at least in part an interior of the laboratory safety enclosure when in a privacy mode thereof.

13. A laboratory safety enclosure assembly comprising:
a laboratory safety enclosure having an interior and including a sash, window and/or door;
a position-sensing device via which positioning of the sash, window and/or door is measured and one or more signals based thereon are emitted;
a display via which access to the interior of the laboratory safety enclosure is enabled by logging therein; and
a processor operatively connected to the position-sensing device and the display, with the processor logging each change in positioning of the sash, window and/or door and the processor logging time-in and time-out information and/or the time at which a logging-in event occurs and the time at which a logging-off event occurs.

14. A laboratory safety enclosure assembly according to claim 13, wherein features of the assembly are accessible on the display upon logging-in with user information, and wherein the processor correlates data so logged with the user information so as to determine user-specific safety information, a user-specific safety score, user-specific efficiency information and/or a user-specific efficiency score.

15. A kit for retrofitting a laboratory safety enclosure to obtain the laboratory safety enclosure assembly of claim 13, the kit comprising:
said position-sensing device connectable to the laboratory safety enclosure so as to measure positioning of the sash, window and/or door in real-time and emit one or more signals based thereon are emitted;
said display connectable to the laboratory safety enclosure, the display being operatively connectable to a servomotor of the sash, window and/or door to enable access to the interior of the laboratory safety enclosure, and/or the display being operatively connectable to a locking mechanism of the sash, window and/or door to enable access to the interior of the laboratory safety enclosure; and
said processor operatively connected to the position-sensing device and the display, with the processor logging each change in positioning of the sash, window and/or door and the processor logging time-in and time-out information and/or the time at which a logging-in event occurs and the time at which a logging-off event occurs.

16. A method of improving safety of a laboratory safety enclosure assembly according to claim 1, the method comprising:
requiring that a person be detected by the laboratory safety enclosure assembly via a person-detecting device as a first condition precedent and/or first safety layer/barrier for obtaining access to the laboratory safety enclosure assembly;
requiring that the person be authorized and/or within a database in a memory of the laboratory safety enclosure assembly as a second condition precedent and/or second safety layer/barrier for obtaining access to the laboratory safety enclosure assembly; and
requiring that the person be certified as a third condition precedent and/or third safety layer/barrier for obtaining access to the laboratory safety enclosure assembly.

17. A method according to claim 16, including requiring that the person view training materials via a display of the laboratory safety enclosure assembly as part of the certification process as a fourth condition precedent and/or fourth safety layer/barrier for obtaining access to the laboratory safety enclosure assembly, and/or requiring that the person satisfactorily pass a test or quiz via the display of the laboratory safety enclosure assembly as part of the certification process as a fifth condition precedent and/or fifth safety layer/barrier for obtaining access to the laboratory safety enclosure assembly.

18. A method of determining a rental rate for a laboratory safety enclosure assembly according to claim 1, the method comprising:
measuring via a position-sensing device positioning of the sash, window and/or door of the laboratory safety enclosure assembly in real-time and emitting one or more signals based thereon;
logging via the position-sensing device each change in positioning of the sash, window and/or door and/or obtaining data via the position sensing device related thereto;
correlating via a processor the data so logged with user information; and
determining the rental rate of the laboratory safety enclosure assembly as a function of and/or proportionate to said data so correlated.

19. A method according to claim 18, wherein positioning of the sash, window and/or door for a period of time which exceeds a predetermined time threshold results in an increased said rental rate and wherein positioning of the sash, window and/or door for a period of time which is less than said predetermined time threshold results in a discounted said rental rate.

20. A method according to claim 18, including adjusting the rental rate of the laboratory safety enclosure assembly so determined based on user-specific safety information and/or a user-specific safety score and/or adjusting the rental rate of the laboratory safety enclosure assembly so determined based on the extent to which one or more alarms are actuated while the user is logged in and/or operating the laboratory safety enclosure assembly.
